(19) United States
(12) Patent Application Publication
(10) Pub. No.: US 2019/0023806 A1
(43) Pub. Date: Jan. 24, 2019

(54) CAMELID HEMOGLOBIN ANTIBODIES AND METHODS OF USE

(71) Applicant: QOOLABS, INC., San Diego, CA (US)

(72) Inventor: Hong QI, San Diego, CA (US)

(73) Assignee: QOOLABS, INC., San Diego, CA (US)

(21) Appl. No.: 15/757,641

(22) PCT Filed: Oct. 20, 2016
(86) PCT No.: PCT/US2016/057942

§ 371 Date: Mar. 5, 2018

Related U.S. Application Data
(60) Provisional application No. 62/244,655, filed on Oct. 21, 2015.

(51) Int. Cl. C07K 16/30; G01N 33/569; C12N 15/62

(52) U.S. Cl. CPC C07K 16/3061; G01N 33/56966; C12N 15/62; G01N 33/533; C07K 2317/60; C07K 2317/92; C07K 2317/22

(57) ABSTRACT

The present disclosure in some aspects relates to hemoglobin (including various hemoglobin variants) polypeptides. In some aspects, the present disclosure further relates to hemoglobin antibodies, including camelid antibodies that specifically bind to hemoglobin, and antibody fragments. The disclosure further relates to methods of detecting an analyte in a sample using a camelid antibody, such as a camelid VHH antibody or fragments thereof. In one aspect, provided herein is a technology platform for isolating highly specific antibodies and applying these antibodies in an immunoassay, such as a lateral flow immunoassay (LFIA). In some aspects, this technology is used to develop hemoglobin variant specific antibodies and to produce LFIA devices for rapid and early diagnosis of a disease.

### STATEMENT REGARDING FEDERALLY SPONSORED RESEARCH OR DEVELOPMENT

This invention was made with the support by National Heart, Lung, and Blood Institute, National Institutes of Health, Grant No. 1R43HL123443-01. The U.S. government may have certain rights.

### FIELD

In some aspects, the present disclosure relates to hemoglobin antibodies, including camelid antibodies that specifically bind to hemoglobin (including various hemoglobin variants), and antibody fragments. The disclosure further relates to methods of detecting an analyte in a sample using a camelid antibody, such as a camelid VHH antibody or fragments thereof.

### BACKGROUND

Sickle cell disease (SCD) and thalassemias are the most common genetic disorders of hemoglobin caused by mutations of the β-globin gene. Occurring mainly in tropical regions, these disorders are spreading to most countries with population migration. According to WHO, over 300,000 babies worldwide are born with severe forms of these diseases annually. As high as 30% of people in several regions in Africa and about 5% of the world's population are carriers of a gene for SCD or thalassaemia (who.int/mediacentre/factsheets/fs308/en/). In the United States, about 8% of African-Americans carry the sickle gene. Significant morbidity and mortality are associated with SCD patients. Chronic anemia, acute chest syndrome, stroke, splenic and renal dysfunction, pain crisis, and susceptibility to bacterial infections are the most common complications, usually caused by vascular obstruction and ischemia. In Sub-Saharan Africa, 50-80% of SCD patients die in childhood. In addition to loss of lives, the health care costs associated with SCD are also significant, with over $1.1 billion estimated cost in the US in 2009, with about 40% of patients having at least one hospital stay (who.int/mediacentre/factsheets/fs308/en/).

A wide range of methods are effective to manage hemoglobin disorders. Some simple procedures include healthy diet and high fluid intake, pain medication, vaccination and antibiotics. More complicated and expensive procedures include blood transfusions, bone-marrow transplant, and even gene therapy. The most cost-effective strategy for reducing the burden of SCD is to complement disease management with prevention programs. Early identification of SCD patients and subsequent provision of comprehensive care will effectively reduce the disease complications and improve life quality and save live. Olujohungbe, A. and J. Howard, The clinical care of adult patients with sickle cell disease, Br J Hosp Med (Lond), 2008, 69(11): p. 616-9. In developed countries, the morbidity and mortality have been reduced due to advances in the diagnosis and management of SCD. In the US, newborn screening for SCD is mandatory in all 50 states. From 1999 through 2002, there was a 42% decrease in sickle cell-related deaths in children younger than 4 years of age. Mvundura, M., et al., Health care utilization and expenditures for privately and publicly insured children with sickle cell disease in the United States, Pediatr Blood Cancer, 2009, 53(4): p. 642-6; and Ashley-Koch, A., Q. Yang, and R. S. Olney, Sickle hemoglobin (HbS) allele and sickle cell disease: a HuGE review, Am J Epidemiol, 2000, 151(9): p. 839-45.

Current SCD diagnostic methods include electrophoresis, high-performance liquid chromatography (HPLC) or DNA analysis. Although reliable and effective, these methods are not suitable for neonatal screening in low resource areas, where SCD is most prevalent. In fact, many children in these areas die in early infancy due to potentially treatable complications of SCD, such as pneumonia and acute anemia. Therefore, there is an urgent need for low-cost and accurate point-of-care diagnostic devices for SCD diagnosis.

### SUMMARY

In one aspect, disclosed herein is an isolated camelid antibody that specifically binds to one or more epitopes within a hemoglobin. In some embodiments, the isolated camelid antibody is derived from a camel, a llama, an alpaca (Vicugna pacos), a vicuña (Vicugna vicugna), or a guanaco (Lama guanicoe). In some aspects, the camel is a dromedary camel (Camelus dromedarius), a Bactrian camel (Camelus bactrianus), or a wild Bactrian camel (Camelus ferus).

In any of the preceding embodiments, the isolated camelid antibody can be a polyclonal antibody, a monoclonal antibody, an antibody fragment or a single-domain heavy-chain (VHH) antibody. In one aspect, the VHH antibody is a llama VHH antibody.

In any of the preceding embodiments, the isolated camelid antibody can specifically bind to one or more epitopes within a vertebrate or a mammalian hemoglobin.

In any of the preceding embodiments, the isolated camelid antibody can specifically bind to one or more epitopes within a non-human mammalian hemoglobin, e.g., a monkey or chimpanzee hemoglobin.

In any of the preceding embodiments, the isolated camelid antibody can specifically bind to one or more epitopes within a human hemoglobin.

In any of the preceding embodiments, the isolated camelid antibody can specifically bind to one or more epitopes within a human embryonic hemoglobin, a human fetal hemoglobin, or a human hemoglobin after birth. In some embodiments, the human embryonic hemoglobin is Gower 1 (ζ₂ε₂), Gower 2 (α₂ε₂), hemoglobin Portland I (ζ₂γ₂) or hemoglobin Portland II (ζ₂β₂). In some embodiments, the human fetal hemoglobin is hemoglobin F (α₂γ₂). In some embodiments, the human hemoglobin after birth is hemoglobin A (α₂β₂), hemoglobin A2 (α₂δ₂) or hemoglobin F (α₂γ₂).

In any of the preceding embodiments, the isolated camelid antibody can specifically bind to one or more epitopes within a mutant of a hemoglobin. In some aspects, the mutant of a hemoglobin is due to amino acid substitution, amino acid deletion and/or amino acid addition.

In any of the preceding embodiments, the isolated camelid antibody can specifically bind to one or more epitopes within a hemoglobin associated with a disease or a disorder. In some aspects, the disease or disorder is hemoglobinopathy. In some aspects, the hemoglobinopathy is a sickle-cell disease (SCD) or thalassemia (or thalassaemia).

In any of the preceding embodiments, the isolated camelid antibody can specifically bind to one or more epitopes within a hemoglobin selected from the group consisting of hemoglobin D-Punjab, (α₂β^{D}₂), hemoglobin H (β₄), hemoglobin Barts, (γ₄), hemoglobin S (α₂β^{S}₂), hemoglobin C (α₂β^{C}₂), hemoglobin E (α₂β^{E}₂), hemoglobin AS and hemoglobin SC.

In any of the preceding embodiments, the isolated camelid antibody can specifically bind to one or more epitopes within a hemoglobin A, hemoglobin A2, hemoglobin C, hemoglobin S, or a combination thereof.

In any of the preceding embodiments, the isolated camelid antibody can specifically bind to one or more epitopes within the polypeptide comprising the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or any combination thereof. In any of the preceding embodiments, the epitope can be between about 3 contiguous amino acid residues, and about 5, about 6, about 7, and up to about 8 to about 10 contiguous amino acids in the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9.

In any of the preceding embodiments, the isolated camelid antibody can be produced by a process that comprises the steps of: a) immunizing a camelid with a polypeptide comprising the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or any combination thereof; and b) recovering the antibody from the camelid. In one aspect, the immunized camelid is a llama.

In any of the preceding embodiments, the isolated camelid antibody can specifically bind to one or more subunits of the hemoglobin, or specifically binds to the hemoglobin. In some aspects, the hemoglobin is a non-human mammalian hemoglobin, e.g., a monkey or chimpanzee hemoglobin. In some aspects, the isolated camelid antibody can specifically bind to a human hemoglobin or one or more subunits thereof. In some embodiments, the isolated camelid antibody specifically binds to a mutant human hemoglobin (or a subunit of thereof) with better specificity and/or affinity than binding to a corresponding wild-type human hemoglobin (or a subunit of thereof). In other embodiments, the isolated camelid antibody specifically binds to a wild-type human hemoglobin (or a subunit of thereof) with better specificity and/or affinity than binding to a corresponding mutant human hemoglobin (or a subunit of thereof).

In some embodiments, the isolated camelid antibody specifically binds to a human hemoglobin (or a subunit of thereof) associated with a disease or a disorder with better specificity and/or affinity than binding to a corresponding human hemoglobin (or a subunit of thereof) not associated with the disease or a disorder. In other embodiments, the isolated camelid antibody specifically binds to a human hemoglobin (or a subunit of thereof) not associated with a disease or a disorder with better specificity and/or affinity than binding to a corresponding human hemoglobin (or a subunit of thereof) associated with the disease or a disorder.

In any of the preceding embodiments, the isolated camelid antibody can be a part of a fusion polypeptide. In some embodiments, the fusion polypeptide comprises a variable region of a camelid antibody and a constant region of a non-camelid antibody. In some embodiments, the fusion polypeptide comprises a variable region of a first camelid antibody and a constant region of a second camelid antibody. In other embodiments, the fusion polypeptide comprises a variable region of a llama antibody and a constant region of a non-camelid antibody. In still other embodiments, the fusion polypeptide comprises a variable region of a llama antibody and a constant region of a rabbit antibody. In some aspects, the fusion polypeptide is a fusion llama VHH antibody that comprises a variable region of the llama VHH antibody and a Fc region of a rabbit antibody.

In any of the preceding embodiments, the isolated camelid antibody can be a humanized antibody.

In any of the preceding embodiments, the isolated camelid antibody can be conjugated to a detectable label. In some embodiments, the detectable label is a colorimetric, a radioactive, an enzymatic, a luminescent or a fluorescent label. In any of the preceding embodiments, the detectable label can be a soluble label or a particle (such as a nanoparticle or a microparticle) or particulate label.

In any of the preceding embodiments, the isolated camelid antibody can be attached to a solid surface, such as a blot, a membrane, a sheet, a paper, a bead, a particle (such as a nanoparticle or a microparticle), an assay plate, an array, a glass slide, a microtiter, or an ELISA plate.

In one aspect, disclosed herein is a method for detecting a hemoglobin polypeptide in a sample, which method comprises contacting the hemoglobin polypeptide in the sample with an isolated camelid antibody of any of the preceding embodiments, and detecting a polypeptide-antibody complex formed between the hemoglobin polypeptide in the sample and the isolated camelid antibody to assess the presence, absence and/or amount of the hemoglobin polypeptide in the sample.

In some embodiments, the sample is from a subject, e.g., a mammal. In some embodiments, the mammal is a human.

In any of the preceding embodiments, the method can be used for diagnosis, prognosis, stratification, risk assessment, or treatment monitoring of a hemoglobin associated disease or a disorder. In one aspect, the disease or disorder is hemoglobinopathy. In another aspect, the hemoglobinopathy is a sickle-cell disease (SCD) or thalassemia (or thalassaemia).

In any of the preceding embodiments, the presence or a normal level of a hemoglobin A, and the absence or a reduced level of hemoglobin C and hemoglobin S can indicate that the mammal does not have a hemoglobin C or hemoglobin S associated disease or a disorder.

In any of the preceding embodiments, the presence or a normal level of a hemoglobin A and a hemoglobin S, and the absence or a reduced level of a hemoglobin C can indicate that the mammal has sickle cell trait (SCT).

In any of the preceding embodiments, the presence or a normal level of a hemoglobin S, and the absence or a reduced level of a hemoglobin A and a hemoglobin C can indicate that the mammal has sickle cell trait (SCT).

In any of the preceding embodiments, the presence or a normal level of a hemoglobin A and a hemoglobin C, and the absence or a reduced level of a hemoglobin S can indicate that the mammal is a hemoglobin C carrier.

In any of the preceding embodiments, the presence or a normal level of a hemoglobin C, and the absence or a reduced level of a hemoglobin A and a hemoglobin S can indicate that the mammal has a hemoglobin C associated disease or disorder.

In any of the preceding embodiments, the presence or a normal level of a hemoglobin C and a hemoglobin S, and the absence or a reduced level of a hemoglobin A can indicate that the mammal has sickle cell disease with S/C mutation and is a hemoglobin C carrier.

In any of the preceding embodiments, the presence or a normal level of a hemoglobin S, the absence or a reduced level of a hemoglobin A and a hemoglobin C, and an elevated level of hemoglobin A2 and/or hemoglobin F can indicate that the mammal has HbS/β⁰ thalassaemia.

In any of the preceding embodiments, the presence or a normal level of a hemoglobin S, the absence or a reduced level of a hemoglobin A and a hemoglobin C, and a normal level of hemoglobin A2 can indicate that the mammal has HbS/β⁺ thalassaemia.

In any of the preceding embodiments, the normal level of a hemoglobin in a subject can be between about 120 g/L and about 175 g/L.

In any of the preceding embodiments, the sample can be selected from the group consisting of a whole blood sample, a serum, a plasma, a urine and a saliva sample.

In any of the preceding embodiments, the sample can be a clinical sample.

In any of the preceding embodiments, the polypeptide-antibody complex can be assessed by a sandwich or competitive assay format. In one aspect, the camelid antibody is attached to a surface and functions as a capture antibody. In another aspect, the camelid antibody is labeled. In some embodiments, the polypeptide-antibody complex is assessed by a sandwich assay format that uses two camelid antibodies, one being a capture antibody and the other being a labeled antibody. In other embodiments, the polypeptide-antibody complex is assessed by a competitive assay format that uses a labeled camelid antibody and a hemoglobin polypeptide, or a fragment or an analog thereof, being a capture reagent.

In any of the preceding embodiments, the polypeptide-antibody complex can be assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, μ-capture assay, inhibition assay and avidity assay.

In any of the preceding embodiments, the polypeptide-antibody complex can be assessed in a homogeneous or a heterogeneous assay format.

In any of the preceding embodiments, the method can further comprise disassociating the hemoglobin polypeptide in the sample from an antibody of the subject to be tested. In one aspect, the hemoglobin polypeptide in the sample is disassociated from the antibody of the subject to be tested by changing the pH of the sample to be 4 or lower, or to be 9 or higher, by treating the sample with a protein denaturing agent, and/or by heating the sample to between about 35° C. and about 95° C., preferably to between about 45° C. and about 70° C., concurrently with or before contacting the sample with the camelid antibody. In another aspect, the protein denaturing agent is guanidine hydrochloride (e.g., about 1 M to about 6 M), guanidinium thiocyanate (e.g., about 1 M to about 6 M), SDS (e.g., about 0.1% to about 2%), β-mercaptoethanol, DTT or other reducing agent for disulfide bond disruption at various concentrations, or urea (e.g., about 2 M to about 8 M), or any combination thereof.

In any of the preceding embodiments, the method can further comprise adjusting the pH of the sample to between about 6 and about 8, and/or removing the protein denaturing agent concurrently with or before contacting the sample with the camelid antibody.

In any of the preceding embodiments of a method disclosed herein, the camelid antibody can be a camelid VHH antibody, and the sample can be contacted with the camelid VHH antibody at a pH that is at 4 or lower, or at 9 or higher, and/or in the presence of the protein denaturing agent. In one aspect, the camelid VHH antibody is a llama VHH antibody.

In any of the preceding embodiments, the hemoglobin polypeptide can be comprised in a subunit of a hemoglobin, or can be comprised in a hemoglobin.

Also disclosed herein is a kit for detecting a hemoglobin polypeptide, and the kit comprises, in a container, an isolated camelid antibody of any of the preceding embodiments. In one aspect, the camelid antibody is labeled, and the kit further comprises a hemoglobin polypeptide, or a fragment or an analog thereof, immobilized on a solid surface.

In one aspect, disclosed herein is a use of a kit of any of the preceding embodiments, for detecting a hemoglobin polypeptide.

In another aspect, disclosed herein is a lateral flow device comprising a matrix that comprises an isolated camelid antibody of any of the preceding embodiments immobilized on the matrix. In one embodiment, the camelid antibody is labeled. In another embodiment, the labeled camelid antibody is configured to be moved by a liquid sample and/or a further liquid to a test site and/or a control site to generate a detectable signal.

In any of the preceding embodiments, the matrix can comprise a hemoglobin polypeptide, or a fragment or an analog thereof, immobilized on a test site.

In yet another aspect, disclosed herein is a use of a lateral flow device of the preceding embodiments for detecting a hemoglobin polypeptide.

In still another aspect, disclosed herein is a polynucleotide which encodes an isolated camelid antibody of any of the preceding embodiments, or a complimentary strand thereof. In one aspect, the polynucleotide is codon-optimized for expression in a non-human organism or a cell. In one embodiment, the organism or cell is a virus, a bacterium, a yeast cell, a plant cell, an insect cell, or a mammalian cell such as a cultured human cell.

In any of the preceding embodiments, the polynucleotide can be DNA or RNA.

In some embodiments, the polynucleotide comprises a nucleotide sequence encoding the amino acid sequence of SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 21. In some embodiments, the polynucleotide comprises a nucleotide sequence encoding an amino acid sequence of at least about 70%, about 75%, about 80%, about 85%, about 90%, about 95%, or about 99%, or 100% sequence identity with SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 21.

Also disclosed herein is a vector comprising the polynucleotide of any of the preceding embodiments. In one aspect, the polynucleotide further comprises a promoter sequence. In any of the preceding embodiments, the polynucleotide can further encode a tag sequence. In any of the preceding embodiments, the polynucleotide can comprise a poly-A sequence. In any of the preceding embodiments, the polynucleotide can comprise a translation termination sequence.

In a further aspect, disclosed herein is a non-human organism or a cell transformed with the vector of any of the preceding embodiments. In one aspect, the non-human organism or cell is a virus, a bacterium, a yeast cell, an insect cell, a plant cell, or a mammalian cell such as a cultured human cell.

In one aspect, disclosed herein is a method of recombinantly making a camelid antibody that specifically binds to an epitope within a hemoglobin, and the method comprises culturing the organism or cell disclosed herein, and recovering the camelid antibody from the organism or cell. In one embodiment, the method further comprises isolating the camelid antibody, optionally by chromatography.

Disclosed herein is also a camelid antibody produced by a method of any of the preceding embodiments. In one aspect, the camelid antibody so produced comprises a native glycosylation pattern. In any of the preceding embodiments, the camelid antibody so produced comprises a native phosphorylation pattern.

### BRIEF DESCRIPTION OF THE DRAWINGS

FIG. 1 shows characterization of VHH antibodies. Panel A shows the purified VHH proteins in the left two lanes are approximately 21 kDa. Panel B shows the VHHs have apparent kD of about 100 pM. Panel C shows the VHHs can be specifically competed by the cognate antigen.

FIG. 2 shows competition lateral flow immunoassay using a VHH-rFc fusion antibody. The left panel shows competition lateral flow immunoassay without Guanidine HCl and SDS containing buffers. The right panel shows results when Guanidine HCl (1M to 5M, strip 2 to 5) and SDS containing buffers were applied to the test strip (strip 6-9).

FIG. 3 shows ELISA results for antibody clones against each variant hemoglobin protein.

FIG. 4 shows affinity of rabbit Fc fusion antibodies to hemoglobin variants.

FIG. 5 shows the comparison of the binding of different clones of monoclonal antibodies to hemoglobin.

FIG. 6 shows results of blood samples directly tested with the purified single domain antibodies specific to normal “A” or sickle mutant “S” hemoglobin.

FIG. 7 shows a sandwich ELISA assay testing 14 blood samples from different patients.

FIG. 8 shows a typical lateral flow immunoassay device.

### DETAILED DESCRIPTION

A detailed description of one or more embodiments of the claimed subject matter is provided below along with accompanying figures that illustrate the principles of the claimed subject matter. The claimed subject matter is described in connection with such embodiments, but is not limited to any particular embodiment. It is to be understood that the claimed subject matter may be embodied in various forms, and encompasses numerous alternatives, modifications and equivalents. Therefore, specific details disclosed herein are not to be interpreted as limiting, but rather as a basis for the claims and as a representative basis for teaching one skilled in the art to employ the claimed subject matter in virtually any appropriately detailed system, structure, or manner. Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can, be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

All publications, including patent documents, scientific articles and databases, referred to in this application are incorporated by reference in their entireties for all purposes to the same extent as if each individual publication were individually incorporated by reference. If a definition set forth herein is contrary to or otherwise inconsistent with a definition set forth in the patents, patent applications, published applications or other publications that are herein incorporated by reference, the definition set forth herein prevails over the definition that is incorporated herein by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

The practice of the provided embodiments will employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polypeptide and protein synthesis and modification, polynucleotide synthesis and modification, polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, Gabriel, Stephens, Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Ausubel et al. eds., Current Protocols in Molecular Biology (1987); T. Brown ed., Essential Molecular Biology (1991), IRL Press; Goeddel ed., Gene Expression Technology (1991), Academic Press; A. Bothwell et al. eds., Methods for Cloning and Analysis of Eukaryotic Genes (1990), Bartlett Publ.; M. Kriegler, Gene Transfer and Expression (1990), Stockton Press; R. Wu et al. eds., Recombinant DNA Methodology (1989), Academic Press; M. McPherson et al., PCR: A Practical Approach (1991), IRL Press at Oxford University Press; Stryer, Biochemistry (4th Ed.) (1995), W. H. Freeman, New York N.Y.; Gait, Oligonucleotide Synthesis: A Practical Approach (2002), IRL Press, London; Nelson and Cox, Lehninger, Principles of Biochemistry (2000) 3rd Ed., W. H. Freeman Pub., New York, N.Y.; Berg, et al., Biochemistry (2002) 5th Ed., W. H. Freeman Pub., New York, N.Y.; D. Weir & C. Blackwell, eds., Handbook of Experimental Immunology (1996), Wiley-Blackwell; Cellular and Molecular Immunology (A. Abbas et al., W.B. Saunders Co. 1991, 1994); Current Protocols in Immunology (J. Coligan et al. eds. 1991), all of which are herein incorporated in their entireties by reference for all purposes.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

### I. Definitions

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a composition refers to any mixture of two or more products, substances, or compounds, including cells. It may be a solution, a suspension, liquid, powder, a paste, aqueous, non-aqueous or any combination thereof.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

As used herein, the term “epitope” or “antigenic determinant” refers to a site on an antigen to which B and/or T cells respond or a site on a molecule against which an antibody will be produced and/or to which an antibody will bind. For example, an epitope can be recognized by an antibody defining the epitope. An epitope can be either a “linear epitope” (where a primary amino acid primary sequence comprises the epitope; typically at least 3 contiguous amino acid residues, and more usually, at least 5, at least 6, at least 7, and up to about 8 to about 10 amino acids in a unique sequence) or a “conformational epitope” (an epitope wherein the primary, contiguous amino acid sequence is not the sole defining component of the epitope). A conformational epitope may comprise an increased number of amino acids relative to a linear epitope, as this conformational epitope recognizes a three-dimensional structure of the peptide or protein. For example, when a protein molecule folds to form a three dimensional structure, certain amino acids and/or the polypeptide backbone forming the conformational epitope become juxtaposed enabling the antibody to recognize the epitope. Methods of determining conformation of epitopes include but are not limited to, for example, x-ray crystallography, two-dimensional nuclear magnetic resonance spectroscopy and site-directed spin labeling and electron paramagnetic resonance spectroscopy. See, for example, Epitope Mapping Protocols in Methods in Molecular Biology, Vol. 66, Glenn E. Morris, Ed. (1996), the disclosure of which is incorporated in its entirety herein by reference.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745.” (“Contact” numbering scheme), Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme), and Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme).

The boundaries of a given CDR or FR may vary depending on the scheme used for identification. For example, the Kabat scheme is based structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Thus, unless otherwise specified, a “CDR” or “complementary determining region,” or individual specified CDRs (e.g., “CDR-H1, CDR-H2), of a given antibody or region thereof, such as a variable region thereof, should be understood to encompass a (or the specific) complementary determining region as defined by any of the aforementioned schemes. For example, where it is stated that a particular CDR (e.g., a CDR-H3) contains the amino acid sequence of a corresponding CDR in a given V_{H} or VL amino acid sequence, it is understood that such a CDR has a sequence of the corresponding CDR (e.g., CDR-H3) within the variable region, as defined by any of the aforementioned schemes.

Likewise, unless otherwise specified, a FR or individual specified FR(s) (e.g., FR-H1, FR-H2), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, or Contact method.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. See, e.g., Kindt et al., Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. In particular embodiments, the antibodies are single-chain antibody fragments comprising a variable heavy chain region and/or a variable light chain region, such as scFvs.

Single-domain antibodies are antibody fragments comprising all or a portion of the heavy chain variable domain or all or a portion of the light chain variable domain of an antibody. In certain embodiments, a single-domain antibody is a camelid single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody.

A “humanized” antibody is an antibody in which all or substantially all CDR amino acid residues are derived from non-human CDRs and all or substantially all FR amino acid residues are derived from human FRs. The term “chimeric” antibody refers to an antibody in which a portion of the heavy and/or light chain is derived from a particular source or species, while the remainder of the heavy and/or light chain is derived from a different source or species.

Among the provided antibodies are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

The terms “polypeptide” and “protein” are used interchangeably to refer to a polymer of amino acid residues, and are not limited to a minimum length. Polypeptides, including the provided antibodies and antibody chains and other peptides, e.g., linkers, the hemoglobin polypeptides, and/or the hemoglobin antibodies, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “hemoglobin” as used herein encompasses “full-length,” unprocessed hemoglobin as well as any form of hemoglobin that results from processing in the cell or in vitro, or any mutation in the cell or in vitro. The term also encompasses naturally occurring variants of hemoglobin, e.g., splice variants or allelic variants.

The terms “anti-hemoglobin antibody” and “an antibody that binds to hemoglobin” refer to an antibody that is capable of binding hemoglobin (or a subunit thereof, or a fragment thereof) with sufficient affinity and/or specificity. In some embodiments, such an antibody is useful as a diagnostic and/or therapeutic agent in targeting hemoglobin. In one embodiment, the extent of binding of an anti-hemoglobin antibody to an unrelated, non-hemoglobin protein or peptide is less than about 10% of the binding of the antibody to hemoglobin as measured, e.g., by a radioimmunoassay (RIA). In certain embodiments, an antibody that binds to hemoglobin has a dissociation constant (Kd) of ≤1 μM, ≤100 nM, ≤10 nM, ≤1 nM, ≤0.1 nM, ≤0.01 nM, or ≤0.001 nM (e.g., 10⁻⁸M or less, from 10⁻⁸ M to 10⁻¹³M, or from 10 M to 10⁻¹³ M). In certain embodiments, an anti-hemoglobin antibody binds to an epitope of a hemoglobin or variant thereof that is conserved among hemoglobin variants. In other embodiments, an anti-hemoglobin antibody binds to an epitope of a hemoglobin or variant thereof, but does not bind or has a less affinity for one or more other hemoglobin molecules.

As used herein, the term “specific binding” refers to the specificity of a binder, e.g., an antibody, such that it preferentially binds to a target, such as a polypeptide antigen. When referring to a binding partner, e.g., protein, nucleic acid, antibody or other affinity capture agent, etc., “specific binding” can include a binding reaction of two or more binding partners with high affinity and/or complementarity to ensure selective hybridization under designated assay conditions. Typically, specific binding will be at least three times the standard deviation of the background signal. Thus, under designated conditions the binding partner binds to its particular target molecule and does not bind in a significant amount to other molecules present in the sample. Recognition by a binder or an antibody of a particular target in the presence of other potential interfering substances is one characteristic of such binding. Preferably, binders, antibodies or antibody fragments that are specific for or bind specifically to a target bind to the target with higher affinity than binding to other non-target substances. Also preferably, binders, antibodies or antibody fragments that are specific for or bind specifically to a target avoid binding to a significant percentage of non-target substances, e.g., non-target substances present in a testing sample. In some embodiments, binders, antibodies or antibody fragments of the present disclosure avoid binding greater than about 90% of non-target substances, although higher percentages are clearly contemplated and preferred. For example, binders, antibodies or antibody fragments of the present disclosure avoid binding about 91%, about 92%, about 93%, about 94%, about 95%, about 96%, about 97%, about 98%, about 99%, and about 99% or more of non-target substances. In other embodiments, binders, antibodies or antibody fragments of the present disclosure avoid binding greater than about 10%, 20%, 30%, 40%, 50%, 60%, or 70%, or greater than about 75%, or greater than about 80%, or greater than about 85% of non-target substances.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

An “immunoconjugate” is an antibody conjugated to one or more heterologous molecule(s), including but not limited to a gold particle, a fluorescent dye or particle, quantum dots, and latex or any other labels, for example, for use in ELISA or lateral flow assays. In some embodiments, the antibody is or is part of an immunoconjugate, in which the antibody is conjugated to one or more heterologous molecule(s).

Conjugates of an antibody and one or more heterologous molecule(s) may be made using any of a number of known protein coupling agents, e.g., linkers, (see Vitetta et al., Science 238:1098 (1987)), WO94/11026. The linker may be a “cleavable linker,” such as acid-labile linkers, peptidase-sensitive linkers, photolabile linkers, dimethyl linkers, and disulfide-containing linkers (Chari et al., Cancer Res. 52:127-131 (1992); U.S. Pat. No. 5,208,020).

An “individual” or “subject” includes a mammal. Mammals include, but are not limited to, domesticated animals (e.g., cows, sheep, cats, dogs, and horses), primates (e.g., humans and non-human primates such as monkeys), rabbits, and rodents (e.g., mice and rats). An “individual” or “subject” may include birds such as chickens, vertebrates such as fish and mammals such as mice, rats, rabbits, cats, dogs, pigs, cows, ox, sheep, goats, horses, monkeys and other non-human primates. In certain embodiments, the individual or subject is a human.

As used herein, a “sample” can be a solution, a suspension, liquid, powder, a paste, aqueous, non-aqueous or any combination thereof.

In some embodiments, the sample is a biological sample. A biological sample of the present disclosure encompasses a sample in the form of a solution, a suspension, a liquid, a powder, a paste, an aqueous sample, or a non-aqueous sample. As used herein, a “biological sample” includes any sample obtained from a living or viral (or prion) source or other source of macromolecules and biomolecules, and includes any cell type or tissue of a subject from which nucleic acid, protein and/or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. In some embodiments, the sample can be derived from a tissue or a body fluid, for example, a connective, epithelium, muscle or nerve tissue; a tissue selected from the group consisting of brain, lung, liver, spleen, bone marrow, thymus, heart, lymph, blood, bone, cartilage, pancreas, kidney, gall bladder, stomach, intestine, testis, ovary, uterus, rectum, nervous system, gland, and internal blood vessels; or a body fluid selected from the group consisting of blood, urine, saliva, bone marrow, sperm, an ascitic fluid, and subfractions thereof, e.g., serum or plasma.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

### II. Sickle Cell Disease (SCD) and Hemoglobin

Sickle cell disease (SCD) and thalassemias are the most common genetic disorders of hemoglobin caused by mutations of the β-globin gene. Current SCD diagnostic methods include electrophoresis, high-performance liquid chromatography (HPLC) or DNA analysis. Although reliable and effective, these methods are not suitable for neonatal screening in low resource areas, where SCD is most prevalent. In fact, many children in these areas die in early infancy due to potentially treatable complications of SCD, such as pneumonia and acute anemia. Therefore, there is an urgent need for low-cost and accurate point-of-care diagnostic devices for SCD diagnosis.

With affordable SCD point-of-care (POC) diagnostics, newborn screening will become possible for more babies born in low resource areas; POC tests can be used to provide early diagnosis to a much larger number of children. Identified patients will be given appropriate acute therapy and given longer term care to reduce the risk of future complications. Therefore, efficient and inexpensive rapid tests for SCD diagnosis will be a key component to save thousands of lives and reduce health care costs in the long run. To fulfill this need, in some aspects, provided herein is a method of using antibody technology to develop lateral flow immunoassay devices for use to identify SCD and carriers in a few minutes from a drop of patient blood. In some embodiments, such POC diagnostic devices have a significant impact in terms of reducing mortality and morbidity related to SCD.

Normal adult human hemoglobin mostly consists of two alpha and two beta chains to form hemoglobin A. Infants produce mostly hemoglobin F, which consists of two alpha and two gamma chains. There are several mutations related to sickle cell disease and thalassemia. Sickle hemoglobin (Hb S) is caused by a single point mutation on the β chain (encoded by HBB gene on chromosome 11) at the sixth amino acid from glutamic acid to valine (E6V). Patients with sickle cell anemia are homozygous for the Hb S mutation (Hb SS) or compound heterozygous for the Hb S mutation and a beta thalassemia mutation (Hb S/β0 thalassemia). A less severe form of SCD is due to coinheritance of Hb S and hemoglobin C in which the glutamic acid at the sixth position is mutated to lysine (E6K). β-thalassaemias mutations on the HBB gene cause a quantitative deficiency in β chain production, and depending on the mutation, can lead to complete absence) (β⁰) or reduced (β⁺) formation of beta chains. Sickle cell disease encompasses sickle cell anemia (Hb SS or Hbs/(β⁰), as well as other compound heterozygous states, in which the patient has one copy of the HbS and one copy of another abnormal hemoglobin, such as sickle-hemoglobin C disease (HbSC), or sickle β thalassaemia (HbS/β⁺).

In some aspects, variants, homologs, or analogs of hemoglobin polypeptides share a high degree of structural identity and homology (e.g., 90% or more homology). In some aspects, a hemoglobin polypeptide contains conservative amino acid substitutions within the hemoglobin peptide sequences described herein or contain a substitution of an amino acid from a corresponding position in a homologue of hemoglobin peptide. In comparisons of protein sequences, the terms, similarity, identity, and homology each have a distinct meaning as appreciated in the field of genetics. Moreover, orthology and paralogy can be important concepts describing the relationship of members of a given protein family in one organism to the members of the same family in other organisms.

Conservative amino acid substitutions can frequently be made in a protein or peptide without altering either the conformation or the function of the protein or peptide. Peptides of the present disclosure can comprise 1, 2, 3, 4, 5, 6, 7, 8, 9, 10, 11, 12, 13, 14, 15 or more conservative substitutions. Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein or peptide. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pKs of these two amino acid residues are not significant. Still other changes can be considered “conservative” in particular environments (see, e.g. pages 13-15 “Biochemistry” 2nd ED. Lubert Stryer ed (Stanford University); Henikoff et al., PNAS, 1992, 89:10915-19; Lei et al., J Biol Chem, 1995, 270(20):11882-86).

Embodiments of the present disclosure include a wide variety of art-accepted variants or analogs of hemoglobin such as polypeptides having amino acid insertions, deletions and substitutions. Hemoglobin polypeptides, including variants thereof, can be made using methods known in the art such as site-directed mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34:315 (1985)), restriction selection mutagenesis (Wells et al., Philos. Trans. R. Soc. London SerA, 317:415 (1986)) or other known techniques can be performed on the cloned DNA to produce variants of the hemoglobin DNA.

In some embodiments, a hemoglobin polypeptide shares about 30%, about 35%, about 40%, about 45%, about 50%, about 55%, about 60%, about 65%, about 70%, about 75%, about 80%, about 85%, about 90%, about 99%, or 100% similarity with the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, or a fragment thereof. Thus, encompassed by the present disclosure are analogs of hemoglobin polypeptides (nucleic or amino acid) that have altered functional (e.g., immunogenic) properties relative to the starting fragment.

A hemoglobin polypeptide of the present disclosure can be generated using standard peptide synthesis technology or using chemical cleavage methods well known in the art. Alternatively, recombinant methods can be used to generate nucleic acid molecules that encode a hemoglobin polypeptide. In one embodiment, nucleic acid molecules provide a means to generate defined fragments of a hemoglobin polypeptide (or variants, homologs or analogs thereof).

In some embodiments, a hemoglobin polypeptide can be conveniently expressed in cells (such as E. coli or 293T cells) transfected with a commercially available expression vector. Modifications of a hemoglobin polypeptide such as covalent modifications are included within the scope of this disclosure. One type of covalent modification includes reacting targeted amino acid residues of a hemoglobin polypeptide with an organic derivatizing agent that is capable of reacting with selected side chains or the N- or C-terminal residues of the hemoglobin polypeptide. Another type of covalent modification comprises altering the native glycosylation pattern of the hemoglobin polypeptide.

In addition to prokaryotes, eukaryotic microbes such as filamentous fungi or yeast are suitable cloning or expression hosts for expressing vectors, including fungi and yeast strains whose glycosylation pathways have been modified to mimic or approximate those in human cells, resulting in the production of a polypeptide or an antibody with a partially or fully human glycosylation pattern. See Gerngross, Nat. Biotech. 22:1409-1414 (2004), and Li et al., Nat. Biotech. 24:210-215 (2006).

Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells; and NSO cells. In some embodiments, the antibody heavy chains and/or light chains may be expressed in yeast. See, e.g., U.S. Publication No. US 2006/0270045 A1. In some embodiments, a particular eukaryotic host cell is selected based on its ability to make desired post-translational modifications to the heavy chains and/or light chains. For example, in some embodiments, CHO cells produce polypeptides that have a higher level of sialylation than the same polypeptide produced in 293 cells.

In some embodiments, a polypeptide or antibody disclosed herein is produced in a cell-free system. Exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

The hemoglobin polypeptide of the present disclosure can also be modified to form a chimeric molecule comprising a hemoglobin polypeptide fused to another, heterologous polypeptide or amino acid sequence. Such a chimeric molecule can be synthesized chemically or recombinantly. In some aspects, a hemoglobin polypeptide in accordance can comprise a fusion of fragments of the hemoglobin sequence (amino or nucleic acid). Such a chimeric molecule can comprise multiples of the same subsequence of the hemoglobin polypeptide. A chimeric molecule can comprise a fusion of a hemoglobin polypeptide with a poly-histidine epitope tag, which provides an epitope to which immobilized nickel can selectively bind, with cytokines or with growth factors. The epitope tag is generally placed at the amino- or carboxyl-terminus of the hemoglobin polypeptide. In an alternative embodiment, the chimeric molecule can comprise a fusion of a hemoglobin polypeptide with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble form of a hemoglobin polypeptide in place of at least one variable region within an Ig molecule. In one embodiment, the immunoglobulin fusion includes the hinge, CH2 and CH3, or the hinge, CH1, CH2 and CH3 regions of an IgG molecule. For the production of immunoglobulin fusions see, e.g., U.S. Pat. No. 5,428,130 issued Jun. 27, 1995.

### III. Antibodies and Lateral Flow Immunoassays

In some embodiments, provided herein are antibodies and lateral flow immunoassays are suitable for SCD POC diagnostics.

In some aspects, provided herein are anti-hemoglobin antibodies, including functional antibody fragments, including those comprising a variable heavy chain. Also provided are molecules containing such antibodies, e.g., fusion proteins and/or recombinant receptors such as chimeric receptors. Among the provided anti-hemoglobin antibodies are antibodies against the hemoglobin. The antibodies include isolated antibodies.

One aspect of the present disclosure provides antibodies that bind to a hemoglobin polypeptide. Preferred antibodies specifically bind to a hemoglobin polypeptide and do not bind (or bind weakly) to peptides or proteins that are not hemoglobin polypeptides. For example, antibodies that bind to a hemoglobin polypeptide can bind the hemoglobin-related proteins such as the homologs or analogs thereof.

Hemoglobin antibodies of the present disclosure are particularly useful in the treatment, diagnosis, diagnostic and prognostic assays, imaging methodologies, and/or prognosis of hemoglobin-related diseases or conditions.

The present disclosure also provides various immunological assays useful for the detection and quantification of hemoglobin. Such assays can comprise one or more hemoglobin antibodies capable of recognizing and binding a hemoglobin polypeptide, as appropriate. These assays are performed within various immunological assay formats well known in the art, including but not limited to various types of radioimmunoassays, enzyme-linked immunosorbent assays (ELISA), enzyme-linked immunofluorescent assays (ELIFA), and the like.

In other aspects, immunological non-antibody assays of the present disclosure also comprise T cell immunogenicity assays (inhibitory or stimulatory) as well as major histocompatibility complex (MHC) binding assays.

Various methods for the preparation of antibodies are well known in the art. For example, antibodies can be prepared by immunizing a suitable mammalian host using a hemoglobin polypeptide or fragment, in isolated or immunoconjugated form (Antibodies: A Laboratory Manual, CSH Press, Eds., Harlow, and Lane (1988); Harlow, Antibodies, Cold Spring Harbor Press, NY (1989)). In addition, fusion proteins of a hemoglobin polypeptide can also be used, such as a hemoglobin GST-fusion protein. In a particular embodiment, a GST fusion protein comprising all or most of the amino acid sequence of SEQ ID NOs: 1-12 is produced, then used as an immunogen to generate appropriate antibodies. In another embodiment, a hemoglobin polypeptide is synthesized and used as an immunogen.

In addition, naked DNA immunization techniques known in the art are used to generate an immune response to the encoded immunogen (for review, see Donnelly et al., 1997, Ann. Rev. Immunol. 15: 617-648). For example, all or a part of a hemoglobin-encoding polynucleotide can be used to generate an immune response to the encoded immunogen, i.e., a hemoglobin polypeptide.

The amino acid sequence of a hemoglobin polypeptide, such as one shown in SEQ ID NOs: 1-12, can be analyzed to select specific regions of the hemoglobin polypeptide for generating antibodies. For example, hydrophobicity and hydrophilicity analyses of the hemoglobin amino acid sequence are used to identify hydrophilic regions in the hemoglobin structure. Regions of the hemoglobin that show immunogenic structure, as well as other regions and domains, can readily be identified using various other methods known in the art, such as Chou-Fasman, Hopp and Woods, Kyte-Doolittle, Janin, Bhaskaran and Ponnuswamy, Deleage and Roux, Garnier-Robson, Eisenberg, Karplus-Schultz, or Jameson-Wolf analysis. Thus, each region identified by any of these programs or methods is within the scope of the present disclosure. Methods for the generation of hemoglobin antibodies are further illustrated by way of the examples provided herein. Methods for preparing a protein or polypeptide for use as an immunogen are well known in the art. Also well known in the art are methods for preparing immunogenic conjugates of a protein with a carrier, such as BSA, KLH or other carrier protein. In some circumstances, direct conjugation using, for example, carbodiimide reagents are used; in other instances linking reagents such as those supplied by Pierce Chemical Co., Rockford, Ill., are effective. Administration of a hemoglobin immunogen is often conducted by injection over a suitable time period and with use of a suitable adjuvant, as is understood in the art. During the immunization schedule, titers of antibodies can be taken to determine adequacy of antibody formation.

Hemoglobin monoclonal antibodies can be produced by various means well known in the art. For example, immortalized cell lines that secrete a desired monoclonal antibody are prepared using the standard hybridoma technology of Kohler and Milstein or modifications that immortalize antibody-producing B cells, as is generally known. Immortalized cell lines that secrete the desired antibodies are screened by immunoassay in which the antigen is a hemoglobin polypeptide. When the appropriate immortalized cell culture is identified, the cells can be expanded and antibodies produced either from in vitro cultures or from ascites fluid.

Reactivity of a hemoglobin antibody with a hemoglobin polypeptide can be established by a number of well-known means, including Western blot, immunoprecipitation, ELISA, and FACS analyses using, as appropriate, a hemoglobin polypeptide, a hemoglobin expressing cells or extracts thereof. A hemoglobin antibody or fragment thereof can be labeled with a detectable marker or conjugated to a second molecule. Suitable detectable markers include, but are not limited to, a radioisotope, a fluorescent compound, a bioluminescent compound, chemiluminescent compound, a metal chelator or an enzyme. Further, bi-specific antibodies specific for two or more hemoglobin epitopes are generated using methods generally known in the art. Homodimeric antibodies can also be generated by cross-linking techniques known in the art (e.g., Wolff et al., Cancer Res. 53: 2560-2565).

In one aspect, because single domain VHH antibodies from camelids are well suited for large scale production of antibodies, single domain VHH antibodies specific for hemoglobin are provided in the present disclosure. In some embodiments, the present disclosure also includes single-chain antibody fragments, typically comprising linker(s) joining two antibody domains or regions, such two or more single domain VHH antibodies (which can be the same or different). The linker typically is a peptide linker, e.g., a flexible and/or soluble peptide linker, such as one rich in glycine and serine. In some aspects, the linkers rich in glycine and serine (and/or threonine) include at least 80, 85, 90, 91, 92, 93, 94, 95, 96, 97, 98, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS) or GGGS (3GS), such as between 2, 3, 4, and 5 repeats of such a sequence.

Currently mice are the most widely used host for generating monoclonal antibodies, but antibody yields are generally low. Rabbits usually generate better immune response than mice for many immunogens. However, technologies to generate monoclonal rabbit antibodies are not as widely available due to limited availability of fusion partners for hybridomas.

Camelids make a type of antibodies with a homodimeric heavy-chain that is devoid of light chains. The antigen-binding sites of these antibodies are located on a single variable domain (VHH), which also has three hypervariable regions as well as increased variability on the framework regions. See Muyldermans et al., Recognition of antigens by single-domain antibody fragments: the superfluous luxury of paired domains, Trends Biochem Sci, 2001, 26(4):230-35. VHHs often have longer CDR1 and CDR3 regions to increase the structural repertoire of the antigen-binding site and compensate for the absence of the VL CDRs. This special structural feature also allows the paratope to be more concentrated over a smaller area so that small hidden epitopes can still be targeted by VHH.

Nonetheless, VHH antibodies tend to target different epitopes from those of regular antibodies. Particularly, camelids are able to produce high affinity VHH antibodies for haptens and peptides which are otherwise difficult to generate from mice or rabbits through conventional antibody production techniques.

Antigen-specific VHHs can be selected using a number of genetic engineering techniques from synthetic or naïve VHH libraries. See Olichon et al., Preparation of a naive library of camelid single domain antibodies, Methods Mol Biol, 2012, 911:65-78. However, these often results in antibodies with lower affinity for small molecules. See Alvarez-Rueda et al., Generation of llama single-domain antibodies against methotrexate, a prototypical hapten, Mol Immunol, 2007, 44(7):1680-90. In addition, stability and yield are often a problem associated with antibodies developed from synthetic libraries. On the other hand, immunizing llamas by repeated subcutaneous injections reliably gives affinity-matured antibodies as in any other animal system (e.g., goat or rabbit).

The size of the library is often a limiting factor for the throughput and efficiency of library screening, especially when large numbers of antibodies need to be generated. In the case of screening a VHH library, it usually involves cloning the VHH repertoire from B lymphocytes into a phage display vector. After several rounds of panning, individual clones with antigen-specific VHH can be identified. This method is more efficient than corresponding techniques to identify antigen binding partners for conventional antibodies in scFv or Fab format, where VH and VL genes are separately cloned and recombined. For example, from 10⁵ B cells, 10⁵ different VHH genes need to be amplified. If however, a library for both VH and VL regions is created, 10⁵ VH genes will need to be joined to 10⁵ different VL genes in 10¹⁰ clones to cover the entire repertoire.

In a related aspect, the present disclosure provides a method of producing a library of expression vectors encoding VH domains of camelid antibodies, said method comprising the steps: a) amplifying regions of nucleic acid molecules encoding VH domains of camelid antibodies to obtain amplified gene segments, each gene segment containing a sequence of nucleotides encoding a VH domain of a camelid antibody, and b) cloning the gene segments obtained in a) into expression vectors, such that each expression vector contains at least a gene segment encoding a VH domain, whereby a library of expression vectors is obtained.

In one embodiment, the nucleic acid amplified in step a) comprises cDNA or genomic DNA prepared from lymphoid tissue of a camelid, said lymphoid tissue comprising one or more B cells, lymph nodes, spleen cells, bone marrow cells, or a combination thereof. In one aspect, peripheral blood lymphocytes (PBLs) or PBMCs can be used as a source of nucleic acid encoding VH domains of camelid antibodies, i.e. there is sufficient quantity of plasma cells (expressing antibodies) present in a sample of PBMCs to enable direct amplification. This is advantageous because PBMCs can be prepared from a whole blood sample taken from the animal (camelid). This avoids the need to use invasive procedures to obtain tissue biopsies (e.g. from spleen or lymph node), and means that the sampling procedure can be repeated as often as necessary, with minimal impact on the animal. For example, it is possible to actively immunize the camelid, remove a first blood sample from the animal and prepare PBMCs, then immunize the same animal a second time, either with a “boosting” dose of the same antigen or with a different antigen, then remove a second blood sample and prepare PBMCs.

Accordingly, a particular embodiment of this method of the present disclosure may involve: preparing a sample containing PBMCs from a camelid, preparing cDNA or genomic DNA from the PBMCs and using this cDNA or genomic DNA as a template for amplification of gene segments encoding VH domains of camelid antibodies.

In one embodiment the lymphoid tissue (e.g. circulating B cells) is obtained from a camelid which has been actively immunized, as described elsewhere herein. However, this embodiment is non-limiting and it is also contemplated to prepare non-immune libraries and libraries derived from lymphoid tissue of diseased camelids, also described elsewhere herein.

Conveniently, total RNA (or mRNA) can be prepared from the lymphoid tissue sample (e.g. peripheral blood cells or tissue biopsy) and converted to cDNA by standard techniques. It is also possible to use genomic DNA as a starting material.

This aspect of the present disclosure encompasses both a diverse library approach, and a B cell selection approach for construction of the library. In a diverse library approach, repertoires of VH and VL-encoding gene segments may be amplified from nucleic acid prepared from lymphoid tissue without any prior selection of B cells. In a B cell selection approach, B cells displaying antibodies with desired antigen-binding characteristics may be selected, prior to nucleic acid extraction and amplification of VH and VL-encoding gene segments.

Various conventional methods may be used to select camelid B cells expressing antibodies with desired antigen-binding characteristics. For example, B cells can be stained for cell surface display of conventional IgG with fluorescently labelled monoclonal antibody (mAb, specifically recognizing conventional antibodies from llama or other camelids) and with target antigen labelled with another fluorescent dye. Individual double positive B cells may then be isolated by FACS, and total RNA (or genomic DNA) extracted from individual cells. Alternatively cells can be subjected to in vitro proliferation and culture supernatants with secreted IgG can be screened, and total RNA (or genomic DNA) extracted from positive cells. In a still further approach, individual B cells may be transformed with specific genes or fused with tumor cell lines to generate cell lines, which can be grown “at will”, and total RNA (or genomic DNA) subsequently prepared from these cells.

Instead of sorting by FACS, target specific B cells expressing conventional IgG can be “panned” on immobilized monoclonal antibodies (directed against camelid antibodies) and subsequently on immobilized target antigen. RNA (or genomic DNA) can be extracted from pools of antigen specific B cells or these pools can be transformed and individual cells cloned out by limited dilution or FACS.

B cell selection methods may involve positive selection, or negative selection.

Whether using a diverse library approach without any B cell selection, or a B cell selection approach, nucleic acid (cDNA or genomic DNA) prepared from the lymphoid tissue is subject to an amplification step in order to amplify gene segments encoding individual VH domains.

Total RNA extracted from the lymphoid tissue (e.g. peripheral B cells or tissue biopsy) may be converted into random primed cDNA or oligo dT primer can be used for cDNA synthesis, alternatively Ig specific oligonucleotide primers can be applied for cDNA synthesis, or mRNA (i.e. poly A RNA) can be purified from total RNA with oligo dT cellulose prior to cDNA synthesis. Genomic DNA isolated from B cells can be used for PCR.

In some aspects, provided herein are methods of producing renewable antibodies against hemoglobin from camelids, specifically llamas. Camelids produce single-domain heavy-chain antibodies (VHH) in addition to conventional antibodies. See Hamers-Casterman et al., Naturally occurring antibodies devoid of light chains, Nature, 1993, 363(6428):446-8. The antigen specific VHHs are the smallest binding units produced by the immune systems. Compared to conventional antibodies, in some aspects, camelid VHHs have advantages which make them a better system for generating renewable antibodies on a large scale.

In some embodiments, the camelid is first immunized with a hemoglobin polypeptide of the present disclosure. The hemoglobin polypeptide can be the full length hemoglobin (or a variant) or a fragment thereof, and can be a fusion protein with one or more tags. In some embodiments, the same animal is immunized a second time (or additional times), either with a “boosting” dose of the same hemoglobin polypeptide or with a different hemoglobin polypeptide. For example, the camelid can be initially immunized with a hemoglobin fragment fused to a tag, and then boosted with a full length hemoglobin and/or a hemoglobin fragment without the tag, or vice versa.

First, Camelid single-domain antibody fragments make the VHHs more suited for construction of large libraries for in vitro display selection systems. See Arbabi Ghahroudi et al., Selection and identification of single domain antibody fragments from camel heavy-chain antibodies, FEBS Lett, 1997, 414(3):521-6. VHH libraries generated from immunized camelids retain full functional diversity, whereas the conventional antibody libraries suffer from diminished diversity due to reshuffling of VL and VH domains during library construction. See Harmsen et al., Properties, production, and applications of camelid single-domain antibody fragments, Appl Microbiol Biotechnol, 2007, 77(1):13-22; Harmsen et al., Llama heavy-chain V regions consist of at least four distinct subfamilies revealing novel sequence features, Mol Immunol, 2000, 37(10):579-90; van der Linden et al., Induction of immune responses and molecular cloning of the heavy chain antibody repertoire of Lama glama, J Immunol Methods, 2000, 240(1-2):185-95; Frenken et al., Isolation of antigen specific llama VHH antibody fragments and their high level secretion by Saccharomyces cerevisiae, J Biotechnol, 2000, 78(1):11-21. In vitro selection systems immediately provide the identity of genes and corresponding sequences of antibodies selected against a particular target. By introducing additional mutations and constructing secondary libraries, antibody affinity and specificity can be further tailored. Usability of these antibodies can be further expanded through modifications by simple subcloning to create fusion products to enzymes, tags, fluorescent proteins or Fc domains. In some aspects, provided herein are fusion VHH antibodies with rabbit Fc, and the functionality of the fusion antibodies in LFIA devices is demonstrated. In some aspects, the uniform Fc domain on antibodies also makes them easier to be applied in multiplexed immunoassays.

Second, by adopting different binding patterns, VHHs can specifically interact with small molecules. See Fanning et al., An anti-hapten camelid antibody reveals a cryptic binding site with significant energetic contributions from a nonhypervariable loop, Protein Sci, 2011, 20(7):1196-207. Small molecules such as herbicides, caffeine, mycotoxins, trinitrotoluene, steroids, and therapeutic drugs have all been successfully used as haptens to generate specific VHHs from both naïve and immunized camelid VHH display libraries. See Yau et al., Selection of hapten-specific single-domain antibodies from a non-immunized llama ribosome display library, J Immunol Methods, 2003, 281(1-2):161-75; Sheedy et al., Selection, characterization, and CDR shuffling of naive llama single-domain antibodies selected against auxin and their cross-reactivity with auxinic herbicides from four chemical families, J Agric Food Chem, 2006, 54(10):3668-78; Ladenson et al., Isolation and characterization of a thermally stable recombinant anti-caffeine heavy-chain antibody fragment, Anal Chem, 2006, 78(13):4501-8; Alvarez-Rueda et al., Generation of llama single-domain antibodies against methotrexate, a prototypical hapten, Mol Immunol, 2007, 44(7):1680-90; Doyle et al., Cloning, expression, and characterization of a single-domain antibody fragment with affinity for 15-acetyl-deoxynivalenol, Mol Immunol, 2008, 45(14):3703-13; Anderson et al., TNT detection using llama antibodies and a two-step competitive fluid array immunoassay, J Immunol Methods, 2008, 339(1):47-54; and Kobayashi et al., “Cleavable” hapten-biotin conjugates: preparation and use for the generation of anti-steroid single-domain antibody fragments, Anal Biochem, 2009, 387(2):257-66. Anti-peptide VHHs have also been successfully generated from immunized camels. See Aliprandi et al., The availability of a recombinant anti-SNAP antibody in VHH format amplifies the application flexibility of SNAP-tagged proteins, J Biomed Biotechnol, 2010, 2010:658954. Therefore, both synthetic peptides and purified proteins may be used as immunogen to guide the immune response to specific epitopes.

Third, single-domain antibody fragments are well expressed in microorganisms and have a high apparent stability and solubility. In some aspects, without much optimization, several milligrams of VHHs can be purified from each liter of bacterial culture. These properties greatly facilitate the production of such antibodies at larger number/quantity at significant lower cost, therefore will further reduce the cost of immunoassays.

In some embodiments, single domain antibodies and their binding to cognate antigens are extremely stable and resistant to high concentrations of denaturant. This property makes it possible to perform specific immunoassays under denaturing conditions. VHH single domain antibodies can be applied in lateral flow immunoassays for rapid detection of antigen in the presence of strong denaturant. Typically, removal of the denaturant from the assay is not necessary with these antibodies. Therefore, in some aspects, these antibodies are used to detect viral antigens directly from body fluid under denaturing conditions, for example, to provide rapid tests for point-of-care (POC) detection.

In one aspect, provided herein is an immunization and in vitro screening platform that is well suited to generate large numbers of high affinity VHH antibodies. In one aspect, provided herein is an immunization and in vitro screening platform for generating high affinity antibodies to hemoglobin.

In some aspects, the provided antibodies have one or more specified functional features, such as binding properties, including binding to particular epitopes, such as epitopes that are similar to or overlap with those of other antibodies, the ability to compete for binding with other antibodies, and/or particular binding affinities.

In some embodiments, such properties are described in relation to properties observed for another antibody, e.g., a reference antibody. For example, in some embodiments, the antibody specifically binds to an epitope that overlaps with the epitope of hemoglobin bound by a reference antibody, such as antibodies that bind to the same or a similar epitope as the reference antibody. In some embodiments, the antibody competes for binding to hemoglobin with the reference antibody. An antibody “competes for binding” to hemoglobin with a reference antibody if it competitively inhibits binding of the reference antibody to hemoglobin, and/or if the reference antibody competitively inhibits binding of the antibody to hemoglobin. An antibody competitively inhibits binding of a reference antibody to an antigen if the presence of the antibody in excess detectably inhibits (blocks) binding of the other antibody to its antigen.

A particular degree of inhibition may be specified. In some embodiments, addition of the provided antibody in excess, e.g., 1-, 2-, 5-, 10-, 50- or 100-fold excess, as compared to the amount or concentration of the reference antibody, inhibits binding to the antigen by the reference antibody (or vice versa). In some embodiments, the inhibition of binding is by at least 50%, and in some embodiments by at least 55%, 60%, 65%, 70%, 75%, 80%, 85%, 90%, 95%, or 99%. In some aspects, the competitive inhibition is as measured in a competitive binding assay (see, e.g., Junghans et al., Cancer Res. 1990:50:1495-1502). Competitive inhibition assays are known and include ELISA-based, flow cytometry-based assays, and RIA-based assays. In some aspects, competitive inhibition assays are carried out by incorporating an excess of an unlabeled form of one of the antibodies and assessing its ability to block binding of the other antibody, which is labeled with a detectable marker, such that degree of binding and reduction thereof can be assessed by detection of the label or marker.

In one embodiment, a llama (male or female) is immunized following an optimized immunization and boost schedule. In one aspect, specific anti-sera titer is determined at 40 days, 60 days, 80 days and 100 days post immunization. In one aspect, three proteins are used for coating the ELISA plates: 1) the immunogen; 2) the recombinant hemoglobin; and 3) a fusion partner. Typically, 96-well plates are coated with antigen as indicated. Following blocking and washing, 1:10 serial diluted anti-sera are added to each well. Dilutions in the range of 1:10,000 to 1:10,000,000 are adequate in most cases. Bound antibodies are detected with HRP-conjugated goat anti-llama antibody. In one aspect, the ELISA tests are carried out with or without a blocker in the binder buffer. In one aspect, positive high titers in both coated plates and reactions are not blocked by the blocker indicate the presence of hemoglobin specific antibodies in the serum. In some embodiments, positive reactions are seen at 60 days and the titer continues to rise afterwards. Production bleed are typically collected on day 80 and 100 when the titer reaches the highest.

In cases where no specific titer is detected on day 60 due to low immune response, a different llama can be immunized. Recombinant antigen of a different source can be used, such as a recombinant protein purified from pichia. In other embodiments, synthetic peptides are used for immunization (after conjugation to KLH) to cover different regions of the hemoglobin.

In one aspect, provided herein is a method for single domain antibody library construction. Peripheral blood mononuclear cells (PBMC) are isolated by Ficoll gradient and total RNA is isolated from these cells. Each production bleed typically results in recovery of ˜5×10⁸ cells from 500 ml of blood. The cell number and integrity are examined under microscope with Trypan Blue staining. PBMC cells are then processed, and VHH libraries are constructed by RT-PCR. Based on bioinformatics analysis and sequencing of VHH clones, sets of primers are designed for reverse transcription and PCR. A phage display vector with His-tag is then used for cloning the VHH library. Typically, >10⁹ independent clones for each library are obtained. One library is constructed for each immunized llama.

In one aspect, provided herein is a method for VHH library screening. Specific high affinity binders are selected according to an optimized in vitro screening protocol. To isolate highly specific antibody clones, two approaches are incorporated in the protocol. First, hemoglobin coated plates and biotin-hemoglobin/streptavidin magnetic beads are used alternatively in subsequent screening steps to prevent the isolation of phage that binds to the plate or magnetic beads non-specifically. For example, biotin-hemoglobin/streptavidin magnetic beads are used for the first round of screening for higher handling volume, since the starting number of phages is the largest during the first round in order to cover the entire library. For the second round of screening, hemoglobin coated plates are used to select phage. Those phages that non-specifically bind on the magnetic beads and are isolated from the first round of screening have a less chance of binding on the plate. This way, the background is dramatically reduced. Second, a fusion partner can be used in the hybridization buffer to block the binding of fusion partner specific antibodies to the plate or beads. The binding conditions for each round of panning/screening can be adjusted to obtain desired clones, including input antigen concentration, input number of phage, detergent concentration, and number of washing steps. Typically, between about 10% and about 50% of clones are positive high affinity binders after three rounds of screening.

To further increase the chance of isolating pairing antibodies for sandwich immunoassays and to improve the efficiency, in one aspect, a recombinant protein can be used to screen the antibody library. When screening the library with the immunogen (2-192 amino acids), the shorter peptide (1-120 amino acids) can be used as a blocker to favor the isolation of antibodies against epitopes outside of the 1-120 amino acids. Antibodies isolated with two different antigens have a better chance to form pair in sandwich immunoassays.

In one aspect, provided herein is a method for high affinity VHH clone isolation. The phage display system disclosed herein has several convenient features. First, by changing culture conditions, the system can be induced to either preferentially display antibodies on phage particles for screening of phage, or secreting soluble antibodies into the culture media for direct ELISA to identify positive clones. By switching host cells, the system can produce soluble VHH proteins for pilot scale purification and characterization without further subcloning. The VHH sequences are flanked by two rare restriction sites that are also built into our expression vector for Fc fusion protein expression. Once positive clones are identified, the VHH sequence can be easily subcloned into an Fc fusion protein expression vector to produce VHH-Fc proteins. Individual clones producing high affinity specific antibodies will be identified by ELISA. The gene sequence of each clone will be analyzed and aligned to each other and other VHH sequences to identify the framework regions and CDRs of each antibody. Typically, high affinity specific binders are amplified through multiple rounds of screening and therefore multiple clones show identical sequences for the same epitope. Based on sequence information, clones with different sequences can be sorted into different groups. Those clones with more significant differences in the CDR can have a different binding epitope on the antigen. One or two amino acid differences in CDR1 or CDR3 can cause variation in the affinity on the same epitope. Antibodies with multiple amino acid differences spanning CDR1 to CDR3 usually bind on different epitopes. These sequence information therefore can be used for selecting pairing antibodies in the next steps.

In one aspect, the affinity and specificity of the VHH antibodies are examined. In one aspect, the antibody is expressed in rabbit Fc fusion format for lateral flow assays. In another aspect, pairing antibodies for sandwich immunoassays are identified. VHH antibody proteins are purified from the positive clones from E. coli culture. Several milligrams of pure VHH protein are usually obtained from each liter of culture. Purity of protein is examined on SDS-PAGE followed by Coomassie blue staining of the gel. Protein concentration is determined with Bradford assay using Bovine Gamma Globulin Standard (e.g., from Pierce, Cat#23212).

In one aspect, polyclonal antibody raised in goat against llama IgG is used in detecting VHH antibodies in ELISA to determine affinities of the antibodies to their cognate antigens. ELISA plates are coated with BSA-peptide conjugates at 1 ng/μl. Serial diluted purified antibodies can be added to antigen coated wells. After washing steps, VHH antibody binding to hemoglobin can be detected by HRP-conjugated goat anti-llama antibody. TMB substrate can be used to develop color signal of the ELISA. The apparent kD for each purified VHH antibody can be obtained by non-linear regression curve fitting. Typically, the goat anti-llama antibody has a kD of about 10 nM to VHHs (measured by ELISA). Although the affinity of the secondary antibody to VHH sets the limit on measurable kD of VHHs to their cognate antigens, this method typically provides a quick ranking of isolated VHH clones without much manipulation.

Specificity of each VHH can be determined by two ELISA methods. VHH antibodies can be used directly in ELISA to detect binding to the hemoglobin and the fusion partner. Those VHHs that bind to the hemoglobin but not the fusion partner can be further tested in competition ELISA. 96-well plates can be coated with hemoglobin, and a blocker can be serial diluted with binding buffer containing VHH (concentration determined by kD analysis) and added to each well. In cases where the antibody is specific to the hemoglobin in the sample, the hemoglobin competes with the coated protein for binding of the VHH; the blocker does not compete for the binding of antibody. A competition/inhibition curve can be constructed to determine the specificity.

Those antibodies perform well in ELISA under both conditions can be selected for further development.

In another aspect, provided herein is a method for production of VHH fusion antibodies, such as VHH-rFc fusion antibodies. In one aspect, rabbit Fc fusion VHHs are produced. Due to the effect of dimerization, the antibody affinity and specificity are usually improved by fusion to Fc fragments. See Aliprandi et al., The availability of a recombinant anti-SNAP antibody in VHH format amplifies the application flexibility of SNAP-tagged proteins, J Biomed Biotechnol, 2010, 2010:658954.

In some embodiments, an E. coli expression system is used to express antibodies, including single domain, Fab, or full length IgG. The system uses a periplasmic secretion signal to direct expressed protein into the reducing environment of periplasm to facilitate disulfide bond formation and keep the antibodies soluble. In some embodiments, multiple VHH-rFc proteins at ˜mg/L scale are produced in shaker flasks. These antibodies are used to conjugate colloidal gold and applied in lateral flow immunoassays (see the Examples). In one aspect, the bacterial expression system provides a renewable and low cost source for unlimited antibodies, therefore is a better choice for applications in rapid tests.

Genes of those VHH clones that give highest affinities and specificities are subcloned into the expression vector with built-in rabbit Fc region containing the hinge, CH2 and CH3 domains. In one aspect, the vectors are designed with compatible restriction sites for single step ligation and subcloning. The resulted fusion proteins (rFc-VHH) can be easily expressed and purified with protein A/G affinity chromatography at large quantities and high purity. Typically, ˜10 mg of each antibody is purified for rapid test devices. Affinity of the fusion antibodies to their antigens can be re-determined using HRP conjugated goat anti-rabbit polyclonal antibodies, which usually is not a limiting factor in affinity measurements using ELISAs.

Specificity of the antibodies is examined with Western-blot following SDS-PAGE of patient samples containing hemoglobin. The specificity and affinity of selected antibodies can be further determined by label-free, real time kinetic assays (e.g., Octet, Forte Bio). Unlike rough estimates of kinetic information from IC50 values obtained via ELISAs, real-time kinetic measurements offer a direct and more realistic depiction of molecular interactions. Kinetic constants such as ka, kd, K_{D} can be determined. Selected antibodies can be analyzed for their specificity and affinity with the Octet instrument and methods.

In the event that the affinity or specificity of the antibodies is not satisfactory, an affinity maturation steps can be carried out to further improve the antibodies. First, screening is done at lower stringencies to select several candidate clones. Based on the sequence of these candidate clones, antibody affinity/specificity maturation can be performed. DNA sequences at selected positions in the complementarity determination region (CDR), usually CDR3 can be randomized or changed in length to create a sub-library. This library can be subjected to screening as described above to identify specific binders. Typically, the affinity maturation procedures yield antibodies with ˜10 to 1000 fold improved affinities.

In one aspect, provided herein is a method for finding pairing antibodies for sandwich ELISA. Typically, pairing antibodies with different binding epitopes on the antigen are used for sandwich ELISA. Sandwich ELISA can be performed using matrix of VHH antibodies. Capture VHH antibodies can be coated on the plate. After blocking and washing, hemoglobin can be added to the plate and can be captured by the VHH antibody. Rabbit Fc fusion VHH can be used as detection antibody, which is further detected with HRP-goat anti-rabbit Fc antibody. The Sandwich ELISA can also be performed in the reverse order: coating VHH-rFc on the plate, and detecting with VHH antibody which is His-tagged, which can be detected with mouse anti-His Tag antibody. With differential/subtractive screening using two different antigens, pairs of antibodies for sandwich ELISA can be identified.

### III. Methods for Detection and Diagnosis

Also provided herein are methods involving use of the provided binding molecules, e.g., antibodies, in detection of hemoglobin, for example, in diagnostic and/or prognostic methods in association with hemoglobin (such as hemoglobin variants or mutations). The methods in some embodiments include incubating a biological sample with the antibody and/or administering the antibody to a subject. In certain embodiments, the contacting is under conditions permissive for binding of the hemoglobin antibody, such as a single domain VHH antibody, to hemoglobin, and detecting whether a complex is formed between the hemoglobin antibody and hemoglobin. Such a method may be an in vitro or in vivo method.

In some embodiments, a sample, such as a cell, tissue sample, lysate, composition, or other sample derived therefrom is contacted with the hemoglobin antibody and binding or formation of a complex between the antibody and the sample (e.g., hemoglobin in the sample) is determined or detected. When binding in the test sample is demonstrated or detected as compared to a reference cell of the same tissue type, it may indicate the presence of an associated disease or condition. In some embodiments, the sample is from human tissues.

Various methods known in the art for detecting specific antibody-antigen binding can be used. Exemplary immunoassays include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. Exemplary labels include radionuclides (e.g. ¹²⁵I, ¹³¹I, ³⁵S, ³H, or ³²P), enzymes (e.g., alkaline phosphatase, horseradish peroxidase, luciferase, or β-glactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). General techniques to be used in performing the various immunoassays noted above are known to those of ordinary skill in the art.

For purposes of diagnosis, the antibodies can be labeled with a detectable moiety including but not limited to radioisotopes, fluorescent labels, and various enzyme-substrate labels know in the art. Methods of conjugating labels to an antibody are known in the art.

In some embodiments, antibodies need not be labeled, and the presence thereof can be detected using a labeled antibody which binds to the antibodies of the present disclosure.

The antibodies of the present disclosure can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987).

The antibodies and polypeptides can also be used for in vivo diagnostic assays, such as in vivo imaging. Generally, the antibody is labeled with a radionuclide (such as ¹¹¹In, ⁹⁹Tc, ¹⁴C, ¹³¹I, ¹²⁵I, or ³H) so that the cells or tissue of interest can be localized in vivo following administration to a subject.

The antibody may also be used as staining reagent in pathology, e.g., using known techniques.

In another aspect, provided herein is a rapid test with quantum dots labeling for a sensitive and quantitative lateral flow immunoassay. Lateral flow immunoassays (LFIA) use specific antibodies to rapidly detect the presence of antigen in test samples. The assay typically can be performed in less than 10 minutes and require no special equipment or highly trained technicians. The manufacturing costs of these tests are also typically very low compared to other platforms. Since the first introduction of LFIA in pregnancy tests, it has been widely used in clinical POC diagnostics and in the drug abuse screening field.

Aside from many technical details in manufacturing a LFIA device, the most important component for a successful LFIA is typically the target specific antibody. In one aspect, the target specific antibody is a llama single domain antibody as described herein. The detection method is also important. Conventional LFIA is an immuno-chromatographic assay using a colloidal gold or latex-labeled antibody for colorimetric detection of targets. These assays are rapid and simple to use, and are most suitable in field screening applications. However, the results are more qualitative in nature and the sensitivity is often limited.

Fluorescent and luminescent labels have been used to improve sensitivity and quantitation range for LFIA. Semiconductor nanocrystals, also known as quantum dots, are a class of light-emitting materials whose electronic characteristics are closely related to the size and shape of the individual crystal. By simply varying the crystal size, quantum dots emit lights in a wide range of wavelengths, or colors that are less prone to overlap than those of organic dyes. A single light source can excite quantum dots of many colors so that multiple targets can be labeled and detected simultaneously. In addition to this multiplexing capability, quantum dots exhibit brilliant colors and long-term photo-stability and are therefore much brighter than organic dyes and retain their glow much longer. Provided here in some embodiments are methods of using quantum dots for developing multiplexed quantitative point-of-care assay devices, for example, devices for quantitative lateral flow assays using quantum dot labeled antibodies to improve the utility of LFIA as a diagnostic platform. For example, a portable QD (quantum dot) reader (e.g., one from Ocean Nanotech, San Diego) can be used at point-of-care locations. In order to further improve the sensitivity of the hemoglobin LFIA tests, in some aspects, quantum dots are used to label the hemoglobin specific antibodies, for example, VHH antibodies specific for a hemoglobin or variant thereof.

In one aspect, single domain VHH antibodies, including hemoglobin antibodies, are generated by immunizing llama with multiple antigens. In some aspects, the affinity and specificity of the antibodies are determined. In other aspects, the antibody is expressed in rabbit Fc fusion format for lateral flow assays. In still other aspects, pairing antibodies for sandwich immunoassays are identified are provided. In some embodiments, the antibodies are used to further develop diagnostic ELISA kits and rapid test LFIA devices.

In some embodiments, provided herein are rapid test devices with LFIA using colloidal gold and quantum dots. In one aspect, VHH with rabbit Fc and its application on rapid test devices are used to develop the rapid test devices. First, a conventional LFIA with colloidal gold labeling is constructed, which can provide a quick estimate of specificity and detection limit. Using the quantum dot labeled antibodies, sandwich LFIA strips can be assembled. With the optimized condition and constructed LFIA, patient samples can be tested and compared to ELISA results.

In one embodiment, VHH-rFc antibodies are used in lateral flow immunoassays to detect a small molecule hapten, such as one of about 126 Dalton. In one embodiment, a conventional LFIA with colloidal gold labeling is constructed. The limit of detection typically reaches 10 to 100 ng/ml or lower. By varying the amount of antibody printed on the strip and antibody to gold ratio, a working condition for test strips can be identified. Recombinant hemoglobin can be tested to determine the LOD of these devices.

In another aspect, using the quantum dot labeled antibodies, LFIA strips can be assembled. The sensitivity of quantum dot labeling is typically ˜100 fold better than those of colloidal gold. Cross linking condition including ratio of antibody to cross linker or QD and overall concentration can be determined. To accommodate the use of these devices under denaturing conditions where regular goat anti-rabbit antibodies fail to bind targets and cannot be used on the control line, a VHH antibody and its target antigen can be used as control. For example, the antigen (or antigen-conjugate) can be printed on the control line and labeled antigen-specific VHH-rFC can be sprayed on the conjugate pad with the labeled hemoglobin antibodies. In one aspect, the antigen-specific VHH-rFc binds its target in the presence of strong denaturant, and therefore serves as a proper control under this condition.

In one aspect, to construct the LFIA, a nitrocellulose membrane is printed with the antigen (or antigen-conjugate) at the control line at 1 mg/ml at 10 μg/cm speed. The test line is printed with capture antibody at 1 mg/ml. Purified VHH-rFc hemoglobin is conjugated to colloidal gold or quantum dots at between about 5 and about 50 μg/ml (actual concentration to be optimized individually) and dried on conjugation pads with conjugate-release buffer. Hemoglobin in various concentrations can be tested on assembled test strips. Detection limit and linear range can be determined for each pair of antibodies.

In one aspect, a nitrocellulose membrane is printed with goat anti-rabbit antibody at the control line. The test line is printed with a capture antibody. Purified VHH-rFc anti-hemoglobin antibody is conjugated to colloidal gold and dried on conjugate pads with conjugate release buffer.

The current gold standard methods for diagnosis of SCD include isoelectric focusing electrophoresis, capillary electrophoresis, high-performance liquid chromatography (HPLC) or DNA analysis. These methods all require expensive equipment and trained technicians to perform. On the other hand, simple and inexpensive solubility tests have poor sensitivity and specificity and are therefore not suitable for screening purposes.

Since the first introduction of LFIA for pregnancy testing, lateral flow immunoassay (LFIA) has been widely used in POC or point-of-use devices. The LFIA can be performed in less than 5 minutes and results can be read without any special instruments. Many LFIA devices offer over 98% sensitivity and accuracy due to the high quality of antibodies. Therefore, the LFIA would be an excellent choice for POC diagnosis of SCD given highly specific antibodies available for each hemoglobin variants.

There were several efforts in the 1970s and 1980s in generating hemoglobin antibodies for identification of HbS in various immunoassays. These antibodies were generated using synthetic peptides as immunogens, and included polyclonal antibodies from horse, rabbits, and monoclonal antibodies from mice. Jensen, Monoclonal Antibodies to Human Hemoglobin S and Cell lines for the production thereof, 1988, U.S. DOE. However, none of the studies on SCD using antibodies were conducted in a systematic way to cover various conditions associated with SCDs due to lack of comprehensive panel of antibodies specific to hemoglobin variants. Nonetheless, these early studies indicated that it is possible to use peptide as immunogen to generate hemoglobin variant specific antibodies from animals. In some aspects herein, hemoglobin variant specific antibodies and methods for generating and using the same are provide, for example, by using antibody engineering technology in combination with llama single domain antibodies.

In some embodiments, provided herein are llama single domain (VHH) antibodies, and methods and devices using the same for POC diagnostics of SCD. In one aspect, hemoglobin variants specific antibodies are derived from camelids, for example, llamas. Camelids produce single-domain heavy-chain antibodies (VHH) in addition to conventional antibodies. Hamers-Casterman, C., et al., Naturally occurring antibodies devoid of light chains, Nature, 1993, 363(6428): p. 446-8; Muyldermans, S., et al., Sequence and structure of VH domain from naturally occurring camel heavy chain immunoglobulins lacking light chains, Protein Eng, 1994, 7(9): p. 1129-35. The antigen specific VHHs are the smallest binding units produced by the immune systems. In some aspects, by constructing antibody phage display libraries and using in vitro screening methods, specific VHH antibodies are obtained and re-engineered to be used in POC diagnostic devices based on LFIA. Compared to conventional antibodies, in some aspects, camelid VHHs have several advantages to make them better suited as antibodies specific to epitopes with minor differences.

First, Camelid single-domain antibody fragments make the VHHs more suited for construction of large libraries for in vitro display selection systems. Arbabi Ghahroudi, M., et al., Selection and identification of single domain antibody fragments from camel heavy-chain antibodies, FEBS Lett, 1997, 414(3): p. 521-6. VHH libraries generated from immunized camelids retain full functional diversity, whereas the conventional antibody libraries suffer from diminished diversity due to reshuffling of VL and VH domains during library construction. Harmsen, M. M. and H. J. De Haard, Properties, production, and applications of camelid single-domain antibody fragments, Appl Microbiol Biotechnol, 2007, 77(1): p. 13-22; Harmsen, M. M., et al., Llama heavy-chain V regions consist of at least four distinct subfamilies revealing novel sequence features, Mol Immunol, 2000, 37(10): p. 579-90; van der Linden, R., et al., Induction of immune responses and molecular cloning of the heavy chain antibody repertoire of Lama glama, J Immunol Methods, 2000, 240(1-2): p. 185-95; and Frenken, L. G., et al., Isolation of antigen specific llama VHH antibody fragments and their high level secretion by Saccharomyces cerevisiae, J Biotechnol, 2000, 78(1): p. 11-21. In one aspect, in vitro selection systems immediately provide the identity of genes and corresponding sequences of antibodies selected against a particular target. By introducing additional mutations and constructing secondary libraries, antibody affinity and specificity can be further tailored. Usability of these antibodies can be further expanded through modifications by simple subcloning to create fusion products to enzymes, tags, fluorescent proteins or Fc domains. In some embodiments, fusion VHH with rabbit Fc is provided, and its functionality is demonstrated in LFIA devices.

Second, by adopting different binding patterns, VHHs can specifically interact with small molecules. Fanning, S. W. and J. R. Horn, An anti-hapten camelid antibody reveals a cryptic binding site with significant energetic contributions from a nonhypervariable loop, Protein Sci, 2011, 20(7): p. 1196-207. Small molecules such as herbicides, caffeine, mycotoxins, trinitrotoluene, steroids, and therapeutic drugs have all been successfully used as haptens to generate specific VHHs from both naïve and immunized camelid VHH display libraries. Yau, K. Y., et al., Selection of hapten-specific single-domain antibodies from a non-immunized llama ribosome display library. J Immunol Methods, 2003. 281(1-2): p. 161-75; Sheedy, C., et al., Selection, characterization, and CDR shuffling of naive llama single-domain antibodies selected against auxin and their cross-reactivity with auxinic herbicides from four chemical families. J Agric Food Chem, 2006. 54(10): p. 3668-78; Ladenson, R. C., et al., Isolation and characterization of a thermally stable recombinant anti-caffeine heavy-chain antibody fragment. Anal Chem, 2006. 78(13): p. 4501-8; Alvarez-Rueda, N., et al., Generation of llama single-domain antibodies against methotrexate, a prototypical hapten. Mol Immunol, 2007. 44(7): p. 1680-90; Doyle, P. J., et al., Cloning, expression, and characterization of a single-domain antibody fragment with affinity for 15-acetyl-deoxynivalenol. Mol Immunol, 2008. 45(14): p. 3703-13; Anderson, G. P. and E. R. Goldman, TNT detection using llama antibodies and a two-step competitive fluid array immunoassay. J Immunol Methods, 2008. 339(1): p. 47-54; and Kobayashi, N., et al., “Cleavable” hapten-biotin conjugates: preparation and use for the generation of anti-steroid single-domain antibody fragments. Anal Biochem, 2009. 387(2): p. 257-66. Anti-peptide VHHs have also been successfully generated from immunized camels. Aliprandi, M., et al., The availability of a recombinant anti-SNAP antibody in VHH format amplifies the application flexibility of SNAP-tagged proteins. J Biomed Biotechnol, 2010. 2010: p. 658954. In some aspects, to make VHHs disclosed herein, synthetic peptide with single amino acid differences is used as immunogen to produce antibodies specific to hemoglobin variants.

Third, single-domain antibody fragments are well expressed in microorganisms and have a high apparent stability and solubility. Without much optimization, several milligrams of VHHs may be purified from each liter of bacterial culture. These properties greatly facilitate the production of such antibodies at larger number/quantity at significant lower cost, therefore will further reduce the cost of LFIA devices.

Compared to other in vitro screening antibody technologies, despite many claims of success, full IgG antibodies in yeast are largely difficult to express at production scale, and the heavy glycosylation always complicate the antibody production and characterization. Regarding the source of antibody repertoire for library construction, synthetic libraries have fixed and limited diversity even with large library size. As a result, many antibodies isolated from synthetic yeast display libraries are non-specifically “sticky,” with poor specificity when tested with samples other than pure antigen. Single frame-worked synthetic full IgGs are also typically unstable. On the other hand, immunized llamas produce antibodies through a natural selection process. These antibodies have been demonstrated to be superior in affinity, specificity, and stability and have been successfully used in LFIA. Single domain antibodies are extremely stable and their binding to antigens are also resistant to strong denaturant. These properties make many immunoassays possible under denaturing conditions when other antibodies will not function. For example, in order to expose the hemoglobin epitope with specific point mutation for the antibodies to bind specifically, the red blood cells may be fully lysed with Guanidine HCl and applied to LFIA when VHH antibodies are used. Typically, this is not possible with conventional antibodies.

In some embodiments, provided herein are VHH antibodies to HbA, HbF, HbS, HbC, HbA2, and antibodies specific to all variants. In some aspects, the antibodies are produced as fusion proteins, for example, with an Fc domain such as a rabbit Fc domain for easy detection on LFIA. Since hemoglobin is an abundant protein in the blood, in some aspects, sensitivity would not be an issue for LFIA even using colloidal gold labeling, in which the test results can be read without any instruments. In one aspect, the resulted test device has features of LFIA: low cost, portable, stable, sensitive and specific, simple to perform and minimally invasive (finger stick), and rapid (˜5-10 minutes). Such LFIA devices can be used for SCD screening in low resource settings.

In some embodiments, provided herein are single domain VHH antibodies specific to HbA, HbS, HbF, HbC and HbA2. In particular embodiments, the VHH antibodies are from a camelid, such as llama. Also provided herein, in some aspects, are antibodies against sequences common to the variants. In some aspects, antibody affinity and specificity to cognate hemoglobin are determined, for example, by ELISA. In some embodiments, provided herein are antibodies in the form of fusion proteins, such as rabbit Fc fusion proteins, for example for LFIA device construction.

In some embodiments, synthetic peptides specifically representing each hemoglobin variant are made. In one aspect, these synthetic peptides are conjugated to a molecule, such as a carrier. In one aspect, the carrier is also a hapten for immunization. Haptens are substances with a low molecular weight such as peptides, small proteins and drug molecules that are generally not immunogenic and require the aid of a carrier protein to stimulate a response from the immune system in the form of antibody production. In some embodiments, the synthetic peptides are conjugated to Keyhole limpet hemocyanin (KLH) for immunization of a camelid such as a llama.

In some embodiments, the hemoglobin variant comprises HbA, HbS, HbC, HbA2, and/or HbF. In some embodiments, the regions of the amino acid sequences selected for immunization are unique to each variant. In some aspects, additional peptides that are common to all variant forms can be selected, for example, beta, delta, and gamma chains. In some embodiments, antibodies to the common peptides are used as standard to control the presence and/or absence and/or amount of any of the hemoglobin isoforms.

In one aspect, provided herein is a camelid antibody, such as a VHH antibody, that specifically binds to an epitope within SEQ ID NO: 1 (VHLTPEEKSAVTAL). In some embodiments, provided herein is a camelid antibody, such as a VHH antibody, that specifically binds to an epitope within SEQ ID NO: 2 (VHLTPVEKSAVTAL). In some embodiments, provided herein is a camelid antibody, such as a VHH antibody, that specifically binds to an epitope within SEQ ID NO: 3 (VHLTPKEKSAVTAL). In some embodiments, provided herein is a camelid antibody, such as a VHH antibody, that specifically binds to an epitope within SEQ ID NO: 4 (VHLTPEEKTAVNAL). In some embodiments, provided herein is a camelid antibody, such as a VHH antibody, that specifically binds to an epitope within SEQ ID NO: 5 (AHHFGKEFTPPVQA). In some aspects, provided herein is a camelid antibody, such as a VHH antibody, that specifically binds to an epitope within SEQ ID NO: 10 (AHHFGKKFTPPVQA). In some aspects, provided herein is a camelid antibody, such as a VHH antibody, that specifically binds to an epitope within SEQ ID NO: 11 (AHHFGKQFTPPVQA). In some aspects, provided herein is a camelid antibody, such as a VHH antibody, that specifically binds to an epitope within SEQ ID NO: 12 (AHHFGKVFTPPVQA). In some aspects, provided herein is a camelid antibody, such as a VHH antibody, that specifically binds to an epitope within SEQ ID NO: 6 (GHFTEEDKATITSL). In some aspects, provided herein is a camelid antibody, such as a VHH antibody, that specifically binds to an epitope within SEQ ID NO: 7 (LGRLLVVYPWTQRFF). In some aspects, provided herein is a camelid antibody, such as a VHH antibody, that specifically binds to an epitope within SEQ ID NO: 8 (GNPKVKAHGKKVL). In some aspects, provided herein is a camelid antibody, such as a VHH antibody, that specifically binds to an epitope within SEQ ID NO: 9 (LSELHCDKLHVDPENF). In some aspects, provided herein is a camelid antibody, such as a VHH antibody, that specifically binds to an epitope on a sequence within SEQ ID NOs: 1-12, but does not bind to an epitope on at least one other sequence within SEQ ID NOs: 1-12.

In one aspect, provided herein is a camelid antibody, such as a VHH antibody, that specifically binds to an epitope within HbA. In one aspect, provided herein is a camelid antibody, such as a VHH antibody, that specifically binds to an epitope within HbS. In one aspect, provided herein is a camelid antibody, such as a VHH antibody, that specifically binds to an epitope within HbC. In one aspect, provided herein is a camelid antibody, such as a VHH antibody, that specifically binds to an epitope within HbD. In one aspect, provided herein is a camelid antibody, such as a VHH antibody, that specifically binds to an epitope within HbA2. In one aspect, provided herein is a camelid antibody, such as a VHH antibody, that specifically binds to an epitope within HbE. In one aspect, provided herein is a camelid antibody, such as a VHH antibody, that specifically binds to an epitope within HbF. In some aspects, provided herein is a camelid antibody, such as a VHH antibody, that specifically binds to an epitope of at least one of HbA, HbS, HbC, HbD, HbA2, HbE and HbF, but does not bind to an epitope of at least one other proteins within HbA, HbS, HbC, HbD, HbA2, HbE and HbF. In one aspect, provided herein is a VHH antibody that specifically binds HbC, but does not cross react with HbA or HbS.

In some embodiments, a VHH antibody disclosed herein comprises the amino acid sequence of SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 21, or a combination thereof. Exemplary antibody clones include:

Clone 172P2E8:
(SEQ ID NO: 13)
QVQLVESGGGLVQDGGSLRLACVASRSTRDINSMGWYRQAPGEQREFVAS
IGWQGATVYADSVEGRFTISRDDAKNTLYLQMNSLKPEDTAVYYCGADWR
TYGYFYWGQGTQVTVS
Clone 172P2D9:
(SEQ ID NO: 14)
QLVESGGGLVQDGDSLRLACAASATTVDINSMGWYRQAPGKQRELVASIN
TRGCTVYTDSVEGRFIIYRDDTKNTLYLQMYSLKSEDTAVYYCGADWRTN
GYFYWGQGTQVIVS
Clone 172G9:
(SEQ ID NO: 15)
QVQLVESGGGLIQDGGSLRLACVASRSTRGINSMGWYRQAPGEQREFVAS
IGWQGATVYADSVEGRFTISRDDAKNTLYLEMNSLNPEDTAVYYCGADWR
TSGYFYWGQGTQVIVS
Clone 172P1D3:
(SEQ ID NO: 16)
QVQLVESGGGLVQAGGSLRLSCAASGRITSYYGVGWFRQAPGKGREFVAV
VTWNAGITFYADSVKGRFTISRDNAKNTVYLQMNGLKPEDTAVYYCAAGV
FRARGYTLSGEYEYWGQGTQVTVS
Clone 175G2:
(SEQ ID NO: 17)
QVQLVESGGGLVQAGGSLRLSCAASGIIFNTHTMAWYRQAPGKQRELVGR
ITFTGRTIYILDAVKGRFSISRNTADNTLTLQMNSLKPEDTAVYYCQTRN
IRNNNENWGQGTQVTVS
Clone 175H11:
(SEQ ID NO: 18)
QVQLVESGGGLVQAGGSLRLSCAASGRTLSRYAISWFRQAPGKEREFVGR
ITWSGSTNIADSVKGRFTISRDNAKNTVYLQMNSLKPEDTAVYYCAAAPY
GTMSYDYWGQGTQVSVS
Clone 175E1:
(SEQ ID NO: 19)
QVQFVESGGGLVQAGGSLRLSCVASGRAFSTYTIGWYRRPPGKQRELVAT
IGGNGNTYYVGSAKGRFTISRDNAKNTVYLQMNSLKPEDTDVYYCNRLGA
LDTWGQGTQVTVS
Clone 173B1:
(SEQ ID NO: 20)
QVQLVESGGGLVQAGGSLRLSCVASGRIFSPNDMGWYRQVPGKQRELVAG
MSSRGFTQYAESMEGRVTISRNNAENTAYLQMNGLQPDDTAVYYCYLWTE
GHNFWGQGTQVTVS
Clone 173B10:
(SEQ ID NO: 21)
QVQLVESGGGLVQAGGSLRLSCVASGKIFSPNDMGWYRQVPGKQRELVAA
MSSRGFTNYAESLTDRFTVSRDNAKNTVYLQMNGLKPDDTAVYYCYLWTA
GDNFWGQGTQVTVS

An alignment is shown below, and the bolded and underlined amino acid residues indicate the CDR sequences:

| | |
|---|---|
| 173B1 | QVQLVESGGGLVQAGGSLRLSCVASGRIFSPNDMGWYRQVPGKQRELVAGMSSRG-FTQY |
| 173B10 | QVQLVESGGGLVQAGGSLRLSCVASGKIFSPNDMGWYRQVPGKQRELVAAMSSRG-FTNY |
| 175E1 | QVQFVESGGGLVQAGGSLRLSCVASGRAFSTYTIGWYRRPPGKQRELVATIGGNG-NTYY |
| 172P1D3 | QVQLVESGGGLVQAGGSLRLSCAASGRITSYYGVGWFRQAPGKGREFVAVVTWNAGITFY |
| 175H11 | QVQLVESGGGLVQAGGSLRLSCAASGRTLSRYAISWFRQAPGKEREFVGRITWSG-STNI |
| 175G2 | QVQLVESGGGLVQAGGSLRLSCAASGIIFNTHTMAWYRQAPGKQRELVGRITFTGRTIYI |
| 172P2E8 | QVQLVESGGGLVQDGGSLRLACVASRSTRDINSMGWYRQAPGEQREFVASIGWQG-ATVY |
| 172G9 | QVQLVESGGGLIQDGGSLRLACVASRSTRGINSMGWYRQAPGEQREFVASIGWQG-ATVY |
| 172P2D9 | --QLVESGGGLVQDGDSLRLACAASATTVDINSMGWYRQAPGKQRELVASINTRG-CTVY |
| | *:*******:* *.****:*.** . :.*:*: **: **:*. : . |
| | CDR1 CDR2 |
| 173B1 | AESMEGRVTISRNNAENTAYLQMNGLQPDDTAVYYCYLWTEGHN---------FWGQGTQ |
| 173B10 | AESLTDRFTVSRDNAKNTVYLQMNGLKPDDTAVYYCYLWTAGDN---------FWGQGTQ |
| 175E1 | VGSAKGRFTISRDNAKNTVYLQMNSLKPEDTDVYYCNRLGALD----------TWGQGTQ |
| 172P1D3 | ADSVKGRFTISRDNAKNTVYLQMNGLKPEDTAVYYCAAGVFRARGYTLSGEYEYWGQGTQ |
| 175H11 | ADSVKGRFTISRDNAKNTVYLQMNSLKPEDTAVYYCAAAPYGT------MSYDYWGQGTQ |
| 175G2 | LDAVKGRFSISRNTADNTLTLQMNSLKPEDTAVYYCQTRNIRNN-------NENWGQGTQ |
| 172P2E8 | ADSVEGRFTISRDDAKNTLYLQMNSLKPEDTAVYYCGADWRTYG-------YFYWGQGTQ |
| 172G9 | ADSVEGRFTISRDDAKNTLYLEMNSLNPEDTAVYYCGADWRTSG-------YFYWGQGTQ |
| 172P2D9 | TDSVEGRFIIYRDDTKNTLYLQMYSLKSEDTAVYYCGADWRTNG-------YFYWGQGTQ |
| | : .*. : *: :.** *:* .*:.:** **** ****** |
| | CDR3 |
| 173B1 | VTVS |
| 173B10 | VTVS |
| 175E1 | VTVS |
| 172P1D3 | VTVS |
| 175H11 | VSVS |
| 175G2 | VTVS |
| 172P2E8 | VTVS |
| 172G9 | VIVS |
| 172P2D9 | VIVS |
| | * ** |

In some embodiments, provided herein is an antibody that comprises one or more of the CDR sequences (CDR1, CDR1, or CDR3) within the amino acid sequence of SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 21.

In some embodiments, provided herein is an isolated polynucleotide encoding an antibody or antigen binding fragment thereof that binds to a hemoglobin or a subunit or fragment thereof, wherein the antibody or antigen binding fragment thereof comprises a variable region comprising complementarity determining regions (CDRs) having the amino acid sequences of the CDRs within the amino acid sequence of SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 21.

In some embodiments, provided herein is an isolated polynucleotide encoding an antibody or antigen binding fragment thereof that binds to a hemoglobin or a subunit or fragment thereof, wherein the antibody or antigen binding fragment thereof comprises a variable region comprising the amino acid sequence of SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 21.

In some embodiments, a cysteine is added to each peptide at the N-terminus for conjugation. In some aspects, the presence, absence, and/or amount of a specific antibody to each variant in the anti-serum is detected or confirmed, for example, by an immunoassay such as ELISA. For example, anti-serum from a llama immunized with a peptide can be tested for binding of that peptide (or a fragment thereof) when other peptides are used in a blocking solution. Positive binding should not be blocked by the presence of the other peptides to indicate presence of the peptide specific antibodies.

When anti-serum titer reaches its maximum for specific antigen, usually around 80-100 days post immunization, peripheral blood mononuclear cells can be isolated and VHH genes can be cloned into phage display vectors. In some embodiments, provided herein are extra conserved sequences and PCR primers for VHH gene amplifications. In one aspect, provided herein are several highly specific VHH antibodies (affinities in the pM range) which would have been missed using published PCR primers.

In some aspects, in vitro screening can be performed using biotinylated peptides specific to each variants and magnetic beads cell sorting. To improve the chance of isolating variant-specific VHH clones, subtractive screening strategies can be employed. For example: To isolate HbA specific antibodies, unlabeled HbS, HbC, HbA2 and HbF peptides can be added in the hybridization buffer to block the binding of VHH antibodies with affinity to these variants so that they can be depleted from the HbA fraction. Similarly, antibodies specific to other variants can be isolated using the rest of peptides as blocking agent. Using this subtractive screening method, in some embodiments, VHH antibodies that specifically bind on small molecule hapten but not the linker used for conjugation of hapten to KLH can be obtained.

VHH coding region of all potential positive clones can be sequenced. Clones with repeated occurrences are usually the result of amplification from high affinity binders during multiple rounds of in vitro screening, therefore are more likely to be specific binders with high affinity. These clones can be selected for VHH antibody purification and characterization to verify their specificity and determine their affinity to cognate antigen with direct ELISA and competition ELISA. Antibodies showing strong binding to cognate peptide, but not to other peptide in direct ELISA, and strong competition by its cognate peptide, but not by other peptides in competition ELISA can be selected. These ELISA can also be performed using banked blood with known hemoglobin disorders. Antibody specificity can be verified by Western blots using these blood samples as well. Since the antibodies are raised against peptides, they are more likely to recognize denatured proteins on SDS-PAGE and Western blot. Specific antibodies should recognize only the cognate hemoglobin, not other proteins or other hemoglobin variants.

Several specific antibodies are normally found for large antigens. In some embodiments, however, provided herein are antibodies specific to single location on a short peptide, which probably only constitute a single epitope, therefore one specific clone for each variant is expected.

In case the animals fail to produce highly specific antibodies, screening can be done at lower stringencies to select several candidate clones. Based on the sequence of these candidate clones, antibody affinity/specificity maturation can be performed. DNA sequences at selected positions in the complementarity determination region (CDR), usually CDR3 can be randomized or changed in length to create a sub-library. This library can be subjected to screening disclosed herein to identify specific binders.

Positive clones identified can be sub-cloned into rabbit Fc fusion protein expression vectors to produce VHH-rFc. In some aspects, about 20 to 50 mg of each antibody can be produced for production and/or testing of LFIA.

Lateral flow immunoassays have been widely used in POC diagnostics for over 25 years. There have been significant advancements in the technology to improve its sensitivity and quantitation range. In some aspects, provided herein are colloidal gold labeled test strips for qualitative assay. Results of these assays can be easily read by naked eye. In some aspects, the majority cases of SCD and sickle traits can be diagnosed in one-step tests. In cases with compound SCD/thalassaemia, parental tests may be needed to get a confirmation diagnosis, for example, by using the LFIA.

In some aspects, provided herein is a method for fluorescent/quantum dot labeling of an antibody. In some aspects, provided herein is a device comprising a labeled antibody, for example, for semi-quantitative or quantitative tests. In some aspects, the tests can differentiate patients with sickle traits from HbS/β-thalassemia in infants, since the quantity of each form of hemoglobin is different in each case. For example, in Sickle trait patients, the fetal hemoglobin (gamma chain) is more than adult hemoglobin (normal β chain, HbA) and more than the sickle hemoglobin (HbS β chain), or gamma >HbA>HbS. In HbS/β-thalassemia, usually the amount of these hemoglobin appears in the order of gamma >HbS>HbA. With quantitative assay, these two cases can be easily differentiated. The quantitative assays are typically more expensive and require a handheld reader.

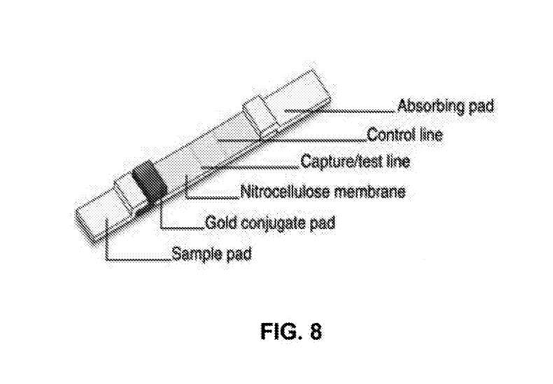

A typical lateral flow immunoassay device is illustrated in FIG. 8. In one aspect, a competitive assay is used with one labeled specific primary antibody (such as colloidal gold) printed on the conjugate pad. The test line is printed with antigen, the control line is printed with secondary antibody to capture the labeled primary antibody. Antigen present in the sample can bind to the primary antibody and compete with the antigen printed on the test line, therefore the intensity of test line signal is inversely correlated with the amount of antigen in the test samples. A sandwich assay uses a labeled primary antibody on the conjugate pad, the test line is printed with another specific antibody that binds to a different epitope on the antigen. Antigen present in the sample can bind on the labeled antibody and be captured by the antibody on the test line. The appearance of the test line typically indicates a positive result.

Table 1 shows possible outcome of test results for various sickle related disorders in infants.

TABLE 1

| | | LFIA device specific to Hb variant | | | | | |
|---|---|---|---|---|---|---|---|
| | | | | | HbA2 | | |
| | Hb variant | | | | (delta | | Hb |
| Disorder | presence | HbA | HbS | HbC | chain) | HbF | common |
| Normal | HbA | ++++ | −−−− | −−−− | + | ++++ | ++++ |
| Sickle Trait | HbS, HbA | ++++ | ++ | −−−− | + | ++++ | ++++ |
| SCD | HbSS | −−−− | ++++ | −−−− | + | ++++ | ++++ |
| HbC carrier | HbA, HbC | ++++ | −−−− | ++++ | + | ++++ | ++++ |
| HbC | HBCC | −−−− | −−−− | ++++ | + | ++++ | ++++ |
| disorder | | | | | | | |
| Sickle | HbSC | −−−− | ++++ | ++++ | + | ++++ | ++++ |
| Trait + C | | | | | | | |
| HbS/β0 | HbS, | −−−− | ++++ | −−−− | ++++ | ++++ | ++++ |
| thalasaemia | Increased | | | | | | |
| | HbA2 | | | | | | |
| HbS β+ | HbS, | ++ | ++++ | −−−− | + | ++++ | ++++ |
| thalasaemia | reduced | | | | | | |
| | HbA | | | | | | |

For most cases there are positive or negative results in one of the LFIA test and the diagnosis would be clear, except in the patients of HbS/β+ thalassemia, who carry one copy of the sickle gene and a reduced expression of β chain from the other copy of the gene. The sample can show positive in HbS and reduced amount of HbA. In cases when HbS/β-thalassemia is suspected, parental tests with these devices can be performed to confirm the diagnosis. The results of SCD and HbS/β0 thalassemia would look similar on HbA and HbS. However, the HbA2 which normally only present in the blood at ≤5% can be increased significantly in HbS/f30 thalassemia. Therefore these two cases could be differentiated by the HbA2 LFIA reading.

Typically, one single drop of blood samples should be enough to perform all the tests since the LFIA is highly sensitive and only require nano to micro grams of hemoglobin which is abundant in the blood (120-175 g/L). The red blood cells can be fully lysed to release and denature the hemoglobin. The sample can be diluted further (estimated in the range of 1:100,000 to achieve 1 μg/ml hemoglobin) before applying on the test strip. The actual dilution factor and buffer can be tested and determined. False-negative and false-positive rates can be determined for each device. Expected performance of LFIA is listed in Table 3 to compare directly to those of HPLC and electrophoresis.

TABLE 3
Comparison of LFIA to HPLC and Electrophoresis.

| | LFIA | HPLC | Electrophoresis |
|---|---|---|---|
| Blood Sample | One drop | 2 ml | One drop. |
| Volume | | | |
| Hb Variants | HbA, HbF, HbA2, HbS, | All normal | All normal hemoglobin and |
| Determined | HbC. Each variant needs | hemoglobin and | variants |
| | one specific antibody to | variants | |
| | be developed | | |
| Instrument | None | HPLC systems | Capillary electrophoresis |
| | | | system |
| Technician | None | Trained/skilled | Trained/skilled |
| requirement | | | |
| Sensitivity | >99% | >99% | >99% |
| Acuracy | >99% | >99% | >99% |
| Time | <10 min | 2 min, +sample prep | 2 days |
| Cost | <$10 with enough | $120 | $150 |
| | volume. | | |

In some embodiments, provided herein is an antibody, such as a camelid VHH antibody, that is highly specific to HbS and/or HbA, and exhibits high sensitivity when there are co-existing conditions such as HbF or severe anemia. In other embodiments, kits and methods of using the antibody are provided, and the test results can be correlated with those of conventional methods including HPLC and electrophoresis.

When the antibodies are ready to be tested and used for lateral flow device prototype production, clinical samples can be used in validating the devices.

### SEQUENCE LISTING

SEQ ID NO: 1
(VHLTPEEKSAVTAL)
SEQ ID NO: 2
(VHLTPVEKSAVTAL)
SEQ ID NO: 3
(VHLTPKEKSAVTAL)
SEQ ID NO: 4
(VHLTPEEKTAVNAL)
SEQ ID NO: 5
(AHHFGKEFTPPVQA)
SEQ ID NO: 6
(GHFTEEDKATITSL)
SEQ ID NO: 7
(LGRLLVVYPWTQRFF)
SEQ ID NO: 8
(GNPKVKAHGKKVL)
SEQ ID NO: 9
(LSELHCDKLHVDPENF)
SEQ ID NO: 10
(AHHFGKKFTPPVQA)
SEQ ID NO: 11
(AHHFGKQFTPPVQA)
SEQ ID NO: 12
(AHHFGKVFTPPVQA)
SEQ ID NO: 13
(QVQLVESGGGLVQDGGSLRLACVASRSTRDINSMGWYRQAPGEQREFVA
SIGWQGATVYADSVEGRFTISRDDAKNTLYLQMNSLKPEDTAVYYCGADW
RTYGYFYWGQGTQVTVS)
SEQ ID NO: 14
(QLVESGGGLVQDGDSLRLACAASATTVDINSMGWYRQAPGKQRELVASI
NTRGCTVYTDSVEGRFIIYRDDTKNTLYLQMYSLKSEDTAVYYCGADWRT
NGYFYWGQGTQVIVS)
SEQ ID NO: 15
(QVQLVESGGGLIQDGGSLRLACVASRSTRGINSMGWYRQAPGEQREFVA
SIGWQGATVYADSVEGRFTISRDDAKNTLYLEMNSLNPEDTAVYYCGADW
RTSGYFYWGQGTQVIVS)
SEQ ID NO: 16
(QVQLVESGGGLVQAGGSLRLSCAASGRITSYYGVGWFRQAPGKGREFVA
VVTWNAGITFYADSVKGRFTISRDNAKNTVYLQMNGLKPEDTAVYYCAAG
VFRARGYTLSGEYEYWGQGTQVTVS)
SEQ ID NO: 17
(QVQLVESGGGLVQAGGSLRLSCAASGIIFNTHTMAWYRQAPGKQRELVG
RITFTGRTIYILDAVKGRFSISRNTADNTLTLQMNSLKPEDTAVYYCQTR
NIRNNNENWGQGTQVTVS)
SEQ ID NO: 18
(QVQLVESGGGLVQAGGSLRLSCAASGRTLSRYAISWFRQAPGKEREFVG
RITWSGSTNIADSVKGRFTISRDNAKNTVYLQMNSLKPEDTAVYYCAAAP
YGTMSYDYWGQGTQVSVS)
SEQ ID NO: 19
(QVQFVESGGGLVQAGGSLRLSCVASGRAFSTYTIGWYRRPPGKQRELVA
TIGGNGNTYYVGSAKGRFTISRDNAKNTVYLQMNSLKPEDTDVYYCNRLG
ALDTWGQGTQVTVS)
SEQ ID NO: 20
(QVQLVESGGGLVQAGGSLRLSCVASGRIFSPNDMGWYRQVPGKQRELVA
GMSSRGFTQYAESMEGRVTISRNNAENTAYLQMNGLQPDDTAVYYCYLWT
EGHNFWGQGTQVTVS)
SEQ ID NO: 21
(QVQLVESGGGLVQAGGSLRLSCVASGKIFSPNDMGWYRQVPGKQRELVA
AMSSRGFTNYAESLTDRFTVSRDNAKNTVYLQMNGLKPDDTAVYYCYLWT
AGDNFWGQGTQVTVS)

### EXAMPLES

### Example 1

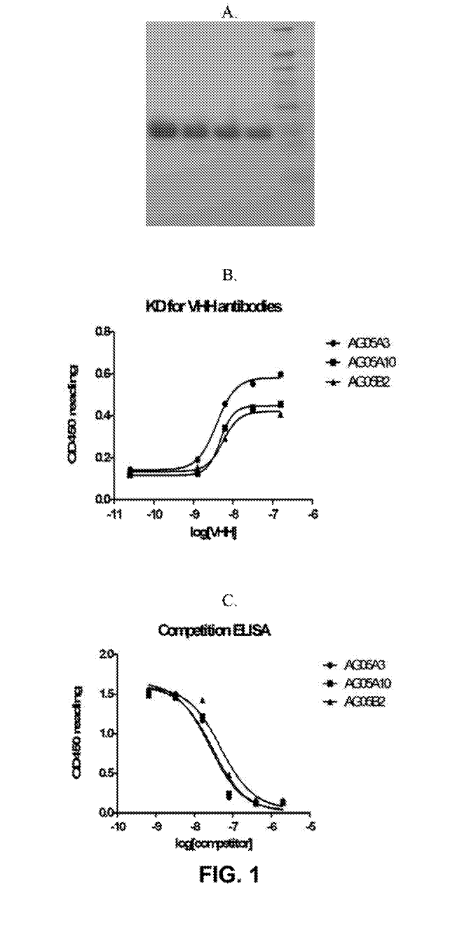

Provided in this example is method for isolating high affinity VHH antibodies from immunized llamas through in vitro screening. Using this method, multiple VHH antibodies for small molecule haptens were isolated. Affinity and specificity of each antibody was determined by direct and competition ELISA. VHH antibodies were purified at milligram scale to >95% purity (FIG. 1A, the purified VHH proteins in the left two lanes were approximately 21 kDa). Many of the selected VHHs have apparent kD of about 100 pM (FIG. 1B), and can be specifically competed by the cognate antigen (FIG. 1C).

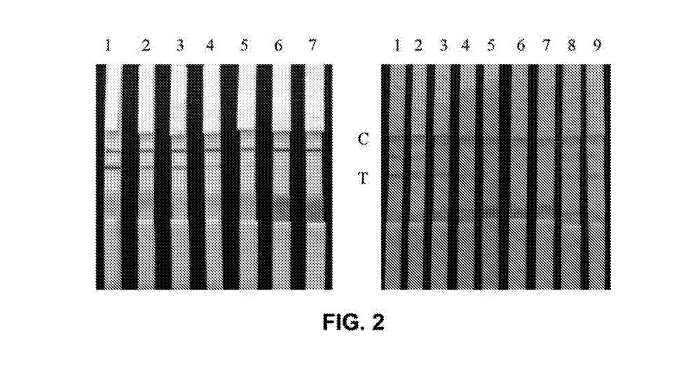

Further, fusion proteins of VHH antibodies with rabbit Fc domains (VHH-rFc) were made. The expressed/purified antibodies can be detected with widely available secondary antibodies to rabbit IgG. These antibodies were used to produce LFIA devices (FIG. 2, left). In addition, the binding of VHH-rFc antibody to its antigen was stable in the presence of up to 3M Guanidine HCl, while goat anti-rabbit IgG failed to bind rabbit IgG at 2M Guanidine HCl. Performing LFIA under strong denaturing condition allows analysis of many proteins that cannot be detected under conventional natural conditions.

FIG. 2 (left) shows competition lateral flow immunoassay using VHH-rFc fusion antibody for AG01. When AGO1 is not present in the sample (strip 1), the labeled VHH-rFc antibody is captured by the AG01-BSA on the test line therefore a visible line appears. When AGO1 is present in the sample at high concentrations (strip 7), the free AGO1 in the sample competes with the AG01-BSA on the test line for the binding of VHH-rFc, therefore the test line is invisible. FIG. 2 (right) shows results when Guanidine HCl (1M to 5M, strip 2 to 5) and SDS containing buffers were applied to the test strip (strip 6-9).

### Example 2

In this example, synthetic peptides specifically represent hemoglobin variants were custom made and conjugated to Keyhole limpet hemocyanin (KLH) for immunization of llamas (Table 2). The sequences for HbA, HbS, HbC, HbA2, and HbF were selected based on publications in which hemoglobin specific antibodies were described, and these regions of the amino acid sequences are unique to each variant. Three additional peptides that are common to all variant forms (beta, delta, and gamma chains) were selected based on multiple sequence alignments. A peptide covering amino acids 115-128 in beta chain was selected to exclude other rare mutations including O-arab (E→K), D-Los Angeles (E→Q) and D-Camperdown (E→V). Immunization were performed by Abcore (Ramona, Calif.). Five llamas were immunized. Peptides #1-4 were separately used to immunize individual llamas. Peptides #5-9 were pooled and used to immunize the 5^{th} llama.

TABLE 2
Selected peptide sequences to represent hemoglobin variants.

| Pep.# | Hemoglobin Form | Selected Peptide Sequence |
|---|---|---|
| 1 | HbA (beta chain, normal 1-14) | VHLTPEEKSAVTAL |
| | | (SEQ ID NO: 1) |
| 2 | HbS (Beta chain, sickle 1-14) | VHLTPVEKSAVTAL |
| | | (SEQ ID NO: 2) |
| 3 | HbC (Beta chain, variant C 1-14) | VHLTPKEKSAVTAL |
| | | (SEQ ID NO: 3) |
| 4 | HbA2 (Delta chain, 1-14) | VHLTPEEKTAVNAL |
| | | (SEQ ID NO: 4) |
| 5 | HbA (beta chain, normal 115-128) | AHHFGKEFTPPVQA |
| | | (SEQ ID NO: 5) |
| | | [E121 in O-arab (E→K), D-Los Angeles (E→Q), |
| | | D-Camperdown (E→V)] |
| 6 | HbF (gamma chain) | GHFTEEDKATITSL |
| | | (SEQ ID NO: 6) |
| 7 | Hb, common 28-42 | LGRLLVVYPWTQRFF |
| | | (SEQ ID NO: 7) |
| 8 | Hb, common 56-68 | GNPKVKAHGKKVL |
| | | (SEQ ID NO: 8) |
| 9 | Hb, common 88-103 | LSELHCDKLHVDPENF |
| | | (SEQ ID NO: 9) |

Presence of specific antibodies to each variant in the anti-serum was confirmed by ELISA before proceeding to VHH gene cloning. When anti-serum titer reached its maximum for specific antigen, peripheral blood mononuclear cells were isolated and VHH genes cloned into phage display vectors. The number 5 llama was sick during the 2nd month of immunization and this group was lost.

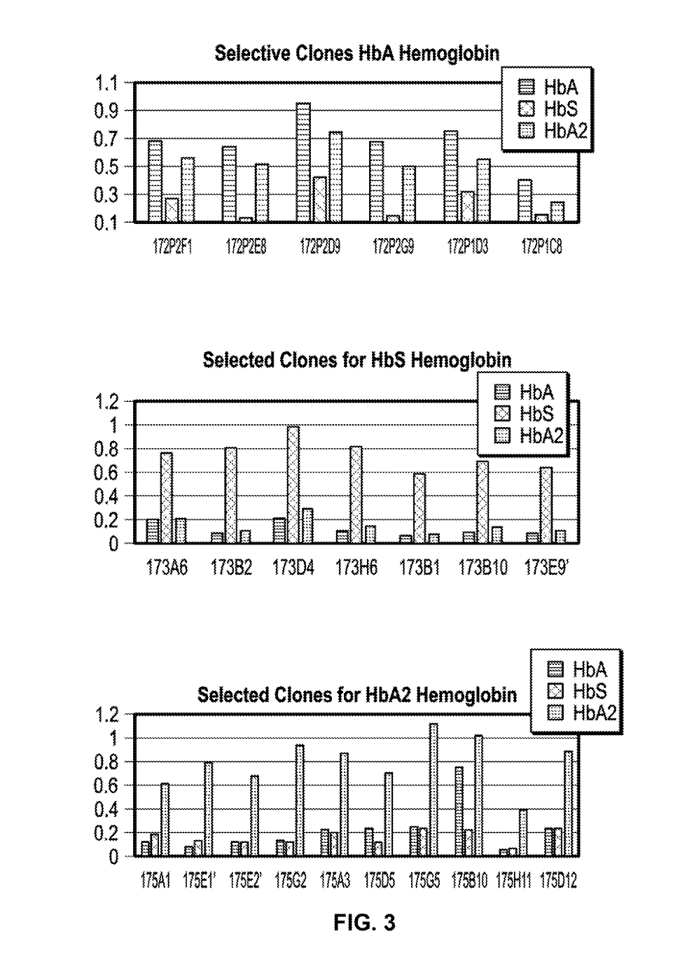

In vitro screening was performed using biotinylated peptides specific to each variants and magnetic beads cell sorting. Multiple positive clones for HbA, HbS, and HbA2 were identified. Different clones showed various affinity and specificity to three forms of hemoglobin (FIG. 3). FIG. 3 shows ELISA results for antibody clones against each variant hemoglobin protein. Hemoglobin was coated on the plate directly and antibodies produced by each clone were applied and detected with HRP goat anti-llama antibody.

For example, the majority clones tested for HbA cross reacted with HbA2 significantly, but cross reacted with HbS to a less degree, especially clone 172P2E8 and 172P2G9. All clones tested for HbS were specific to HbS, with minimal cross reactivity to HbA or HbA2. All clones tested for HbA2 were specific to HbA2, with minimal cross reactivity to HbA or HbS. Clones that do not cross react with HbA2 can be identified. In addition, antibodies cross react with both HbA and HbA2 (but not HbS) can be used to positively identify HbA, since HbA2 (delta chain) present in the adult blood in less than 3%, significantly lower than HbA, HbS or HbC. On the other hand, clones that react with HbA2 but not react with HbA were identified, for use to positively identify HbA2 to compliment the HbA tests.

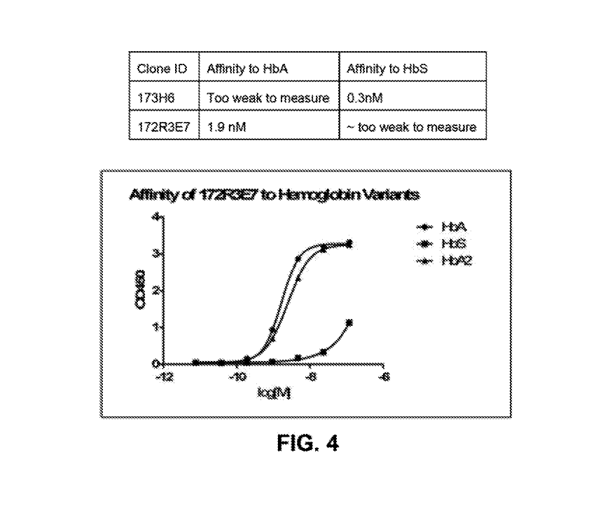

Antibodies as rabbit and llama Fc fusion proteins were produced. The affinity of two of the clones specific to HbA and HbS were determined by ELISA (FIG. 4). Clone 172R3E7 showed an affinity to HbA of 1.9 nM, its affinity to HbA2 was about 2.4 nM, and its affinity to HbS was too weak to measure. Clone 173H6 had an affinity to HbS of 0.3 nM. Its affinity to HbA or HbA2 was too weak to measure. These antibodies are being produced in large amount to be used in lateral flow rapid test to positively identify wild type HbA and sickle cell HbS.

FIG. 4 shows affinity of rabbit Fc fusion antibodies to hemoglobin variants. Hemoglobin was directly coated on ELISA plates, each antibody was serial diluted and applied on the plate followed by detection with HRP-goat-anti-rabbit IgG.

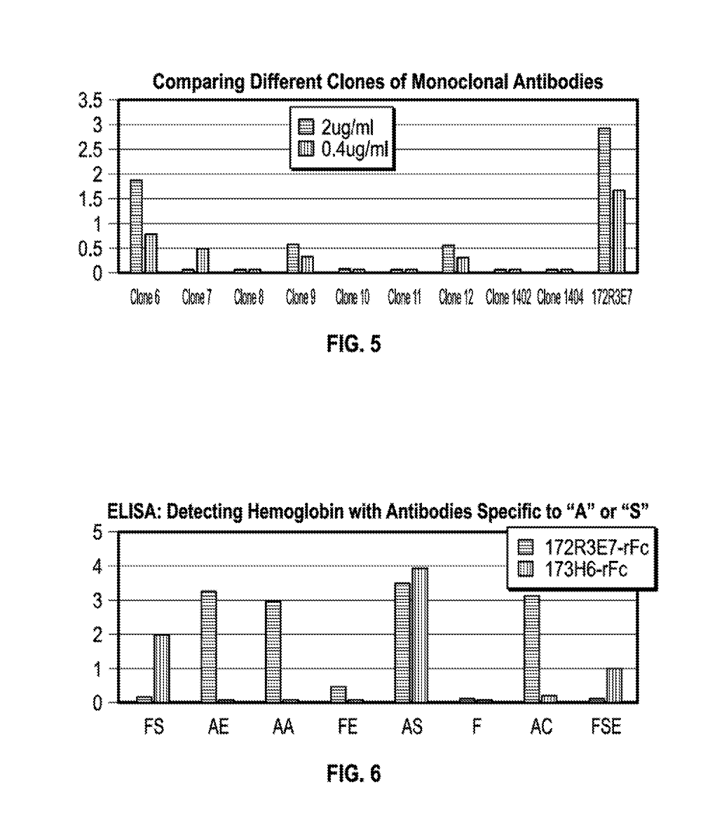

The affinities of these antibodies to nine commercially available monoclonal antibodies were compared. The commercial antibodies are used in diagnostic products by several manufacturers. Five of the commercial antibodies were not reacting with the wild type hemoglobin at 2 μg/ml dilution of antibody when the hemoglobin was coated on the plate directly. Three of the other commercial antibodies reacted with the hemoglobin with weaker affinity than the selected single domain antibody (clone 172R3E7) (FIG. 5). FIG. 5 shows the comparison of the binding of different clones of monoclonal antibodies to hemoglobin. Clones 6 to 12 were from one vendor, clone 1402 and 1404 were from another vendor, all antibodies were claimed to be against hemoglobin. 172R3E7 was a single domain antibody. Results from two concentrations of antibodies were shown (2 μg/ml and 0.4 μg/ml).

Furthermore, clone 6 reacted with the HbS with less affinity than that of HbA, none of the other commercial antibodies reacted with “S” hemoglobin.

Sandwich ELSIA and lateral flow assay test strips for rapid detection of “S” and “A” hemoglobin can be developed by finding/optimizing pairing antibodies.

### Example 3

In this example, blood samples of various mutations were obtained from Oakland Childrens Hospital, assisted by Dr. Hoppe. These blood samples were directly tested with the purified single domain antibodies specific to normal “A” or sickle mutant “S” hemoglobin (FIG. 6). The blood “AE”, “AA”, “AC” reacted strongly with 172R3E7 antibody, “FS” reacted strongly with 173H6, “AS” blood reacted with both antibodies equally well. Blood “FSE” reacted with 173H6 weakly, but not to 172R3E7. This result strongly suggested that the antibodies 173H6 and 172R3E7 may be used in combination to determine the presence of “A” or “S” hemoglobin. Antibodies specific to “C” mutant can be isolated, which may also be used together with the above describe antibodies to positively identify the presence of “C” mutant hemoglobin.

In FIG. 6, whole blood was diluted 100 times with PBS and frozen/thawed to release the hemoglobin. The diluted blood was pre-absorbed with protein A resin to remove human IgG, diluted 100 times further and coated on the ELISA plate directly in PBS. The hemoglobin was then incubated with purified antibody 172R3E7 (specific to normal “A” hemoglobin) and 173H6 (specific to sickle “S” hemoglobin), followed by detection with HRP-goat anti-rabbit IgG.

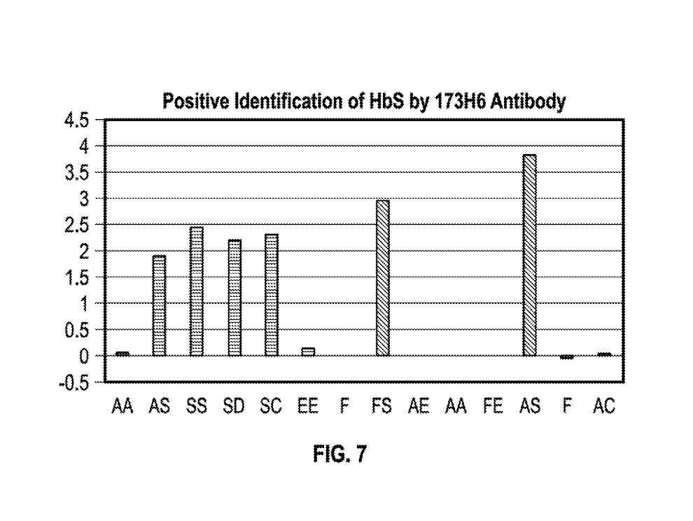

The selected single domain antibodies were tested in sandwich ELISA assays. The 173H6 antibody was able to pair with the mouse monoclonal clone 6 for positive detection of HbS from whole blood (FIG. 7). 14 blood samples with known genotype of hemoglobin were tested with this pair of antibodies. All samples with HbS were highly positive, and samples without HbS were all negative in the ELISA. The presence of HbF or HbC or other mutations did not interfere with the reaction. A monoclonal antibody that pairs with 172R3E7 can be identified to positively detect HbA from whole blood.

FIG. 7 shows a sandwich ELISA assay testing 14 blood samples from different patients. Antibody 173H6 (rabbit Fc fusion) was coated on the plate. Whole blood was diluted and applied on the plate, clone 6 antibody was applied and detected by HRP-goat anti-mouse antibody. Samples were tested in two separate experiments.

1. An isolated camelid antibody that specifically binds to an epitope within a hemoglobin.

2. The isolated camelid antibody of claim 1, which is derived from a camel, a llama, an alpaca (Vicugnapacos), a vicuña (Vicugna vicugna), or a guanaco (Lama guanicoe), optionally wherein the camel is a dromedary camel (Camelus dromedarius), a Bactrian camel (Camelus bactrianus), or a wild Bactrian camel (Camelus ferus).

3. (canceled)

4. The isolated camelid antibody of claim 1, which is a polyclonal antibody, a monoclonal antibody, an antibody fragment, or a single-domain heavy-chain (VHH) antibody.

5. The isolated camelid antibody of claim 4, wherein the VHH antibody is a llama VHH antibody and specifically binds to an epitope within a vertebrate or a mammalian hemoglobin (e.g., a monkey or chimpanzee hemoglobin, or a human hemoglobin).

6-8. (canceled)

9. The isolated camelid antibody of claim 5, which specifically binds to an epitope within a human embryonic hemoglobin (e.g., Gower 1 (ζ₁δ₂), Gower 2 (α₂δ₂), hemoglobin Portland I (ζ₂β₂), or hemoglobin Portland II (ζ₂β₂)), a human fetal hemoglobin (e.g., hemoglobin F (α₂γ₂)), or a human hemoglobin after birth (e.g., hemoglobin A (α₂β₂), hemoglobin A2 (α₂δ₂) or hemoglobin F (α₂γ₂)).

10-12. (canceled)

13. The isolated camelid antibody of claim 1, which specifically binds to an epitope within a mutant of a hemoglobin, or an epitope within a hemoglobin associated with a disease or a disorder.

14-15. (canceled)

16. The isolated camelid antibody of claim 13, wherein the disease or disorder is hemoglobinopathy, e.g., a sickle-cell disease (SCD) or thalassemia (or thalassaemia), optionally wherein the isolated camelid antibody specifically binds to an epitope within a hemoglobin selected from the group consisting of hemoglobin D-Punjab, (α₂β^{D}₂), hemoglobin H (β₄), hemoglobin Barts, (γ₄), hemoglobin S (α₂β^{S}₂), hemoglobin C (α₂β^{C}₂), hemoglobin E (α₂β^{E}₂), hemoglobin AS, and hemoglobin SC.

17-18. (canceled)

19. The isolated camelid antibody of claim 1, which specifically binds to an epitope within a hemoglobin A, hemoglobin A2, hemoglobin C, hemoglobin S, or a combination thereof.

20. The isolated camelid antibody of claim 1, which specifically binds to an epitope within the polypeptide comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or any combination thereof.

21-25. (canceled)

26. The isolated camelid antibody of claim 1, which specifically binds to a mutant human hemoglobin, or a subunit of thereof, with better specificity and/or affinity than binding to a corresponding wild-type human hemoglobin, or a subunit of thereof, or
which specifically binds to a wild-type human hemoglobin, or a subunit of thereof, with better specificity and/or affinity than binding to a corresponding mutant human hemoglobin, or a subunit of thereof.

27. The isolated camelid antibody of claim 1, which specifically binds to a human hemoglobin, or a subunit of thereof, associated with a disease or a disorder with better specificity and/or affinity than binding to a corresponding human hemoglobin, or a subunit of thereof, not associated with the disease or a disorder, or
which specifically binds to a human hemoglobin, or a subunit of thereof, not associated with a disease or a disorder with better specificity and/or affinity than binding to a corresponding human hemoglobin, or a subunit of thereof, associated with the disease or a disorder.

28. The isolated camelid antibody of claim 1, which is a part of a fusion polypeptide comprising a variable region of a camelid (e.g., llama) antibody and a constant region of a non-camelid (e.g., rabbit) antibody or a camelid antibody, optionally wherein the fusion polypeptide is a fusion llama VHH antibody that comprises a variable region of the llama VHH antibody and a Fc region of a rabbit antibody.

29-32. (canceled)

33. The isolated camelid antibody of claim 1, which is a humanized antibody.

34. The isolated camelid antibody of claim 1, which is conjugated to a detectable label, e.g., a colorimetric label, a radioactive label, an enzymatic label, a luminescent label, a fluorescent label, or a soluble label or a particle (such as a nanoparticle or a microparticle), or a particulate label,
optionally wherein the isolated camelid antibody is attached to a solid surface, such as a blot, a membrane, a sheet, a paper, a bead, a particle (such as a nanoparticle or a microparticle), an assay plate, an array, a glass slide, a microtiter, or an ELISA plate.

35-37. (canceled)

38. A method for detecting a hemoglobin polypeptide in a sample, which method comprises contacting the hemoglobin polypeptide in the sample with an isolated camelid antibody of claim 1, and detecting a polypeptide-antibody complex formed between the hemoglobin polypeptide in the sample and the isolated camelid antibody to assess the presence, absence and/or amount of the hemoglobin polypeptide in the sample.

39-40. (canceled)

41. The method of claim 38, wherein the method is used for diagnosis, prognosis, stratification, risk assessment, or treatment monitoring of a hemoglobin associated disease or a disorder, such as hemoglobinopathy, e.g., a sickle-cell disease (SCD) or thalassemia (or thalassaemia), optionally wherein:
the presence or a normal level of a hemoglobin A, and the absence or a reduced level of hemoglobin C and hemoglobin S indicate that the mammal does not have a hemoglobin C or hemoglobin S associated disease or a disorder;
the presence or a normal level of a hemoglobin A and a hemoglobin S, and the absence or a reduced level of a hemoglobin C indicate that the mammal has sickle cell trait (SCT);
the presence or a normal level of a hemoglobin S, and the absence or a reduced level of a hemoglobin A and a hemoglobin C indicate that the mammal has sickle cell trait (SCT);
the presence or a normal level of a hemoglobin A and a hemoglobin C, and the absence or a reduced level of a hemoglobin S indicate that the mammal is a hemoglobin C carrier;
the presence or a normal level of a hemoglobin C, and the absence or a reduced level of a hemoglobin A and a hemoglobin S indicate that the mammal has a hemoglobin C associated disease or disorder;
the presence or a normal level of a hemoglobin C and a hemoglobin S, and the absence or a reduced level of a hemoglobin A indicate that the mammal has sickle cell disease with S/C mutation and is a hemoglobin C carrier;
the presence or a normal level of a hemoglobin S, the absence or a reduced level of a hemoglobin A and a hemoglobin C, and an elevated level of hemoglobin A2 and/or hemoglobin F indicate that the mammal has HbS/β⁰ thalassaemia, or
the presence or a normal level of a hemoglobin S, the absence or a reduced level of a hemoglobin A and a hemoglobin C, and a normal level of hemoglobin A2 indicate that the mammal has HbS/β⁺ thalassaemia.

42-60. (canceled)

61. The method of claim 38, which further comprises disassociating the hemoglobin polypeptide in the sample from an antibody of the subject to be tested.

62. The method of claim 61, wherein the hemoglobin polypeptide in the sample is disassociated from the antibody of the subject to be tested by changing the pH of the sample to be 4 or lower, or to be 9 or higher, by treating the sample with a protein denaturing agent, and/or by heating the sample to between about 35° C. and about 95° C., preferably to between about 45° C. and about 70° C., concurrently with or before contacting the sample with the camelid antibody,
wherein the protein denaturing agent is guanidine hydrochloride (e.g., about 1 M to about 6 M), guanidinium thiocyanate (e.g., about 1 M to about 6 M), SDS (e.g., about 0.1% to about 2%), β-mercaptoethanol, DTT or other reducing agent for disulfide bond disruption at various concentrations, or urea (e.g., about 2 M to about 8 M), or any combination thereof.

63. (canceled)

64. The method of claim 62, which further comprises adjusting the pH of the sample to between about 6 and about 8, and/or removing the protein denaturing agent concurrently with or before contacting the sample with the camelid (e.g., llama) antibody.

65. The method of claim 62, wherein the camelid antibody is a camelid (e.g., llama) VHH antibody, and the sample is contacted with the camelid VHH antibody at a pH that is at 4 or lower, or at 9 or higher, and/or in the presence of the protein denaturing agent.

66-67. (canceled)

68. A kit for detecting a hemoglobin polypeptide, which kit comprises, in a container, an isolated camelid antibody of claim 1, wherein optionally the kit further comprises a hemoglobin polypeptide, or a fragment or an analog thereof, immobilized on a solid surface.

69-70. (canceled)

71. A lateral flow device comprising a matrix that comprises an isolated camelid antibody of claim 1 immobilized on the matrix, wherein optionally the camelid antibody is labeled, and optionally the labeled camelid antibody is configured to be moved by a liquid sample and/or a further liquid to a test site and/or a control site to generate a detectable signal.

72-75. (canceled)

76. A polynucleotide which encodes an isolated camelid antibody of claim 28, wherein the polynucleotide comprises a nucleotide sequence encoding an amino acid sequence of at least about 70%, about 75%, about 80%, about 85%, about 90%, about 95%, about 99%, or 100% sequence identity with SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 21;
or a complimentary strand thereof.

77-80. (canceled)

81. A vector comprising the polynucleotide of claim 76.

82-85. (canceled)

86. A non-human organism or a cell transformed with the vector of claim 81, which is a virus, a bacterium, a yeast cell, an insect cell, a plant cell, or a mammalian cell such as a cultured human cell.

87. (canceled)

88. A method of recombinantly making a camelid antibody that specifically binds to an epitope within a hemoglobin, which method comprises culturing the organism or cell of claim 86, and recovering said camelid antibody from said organism or cell.

89-92. (canceled)


SEQUENCE LISTING (21 sequences)

SEQ ID NO: 1 - protein, 14 aa, Lama glama
VHLTPEEKSAVTAL

SEQ ID NO: 2 - protein, 14 aa, Lama glama
VHLTPVEKSAVTAL

SEQ ID NO: 3 - protein, 14 aa, Lama glama
VHLTPKEKSAVTAL

SEQ ID NO: 4 - protein, 14 aa, Lama glama
VHLTPEEKTAVNAL

SEQ ID NO: 5 - protein, 14 aa, Lama glama
AHHFGKEFTPPVQA

SEQ ID NO: 6 - protein, 14 aa, Lama glama
GHFTEEDKATITSL

SEQ ID NO: 7 - protein, 15 aa, Lama glama
LGRLLVVYPWTQRFF

SEQ ID NO: 8 - protein, 13 aa, Lama glama
GNPKVKAHGKKVL

SEQ ID NO: 9 - protein, 16 aa, Lama glama
LSELHCDKLHVDPENF

SEQ ID NO: 10 - protein, 14 aa, Lama glama
AHHFGKKFTPPVQA

SEQ ID NO: 11 - protein, 14 aa, Lama glama
AHHFGKQFTPPVQA

SEQ ID NO: 12 - protein, 14 aa, Lama glama
AHHFGKVFTPPVQA

SEQ ID NO: 13 - protein, 116 aa, Lama glama
QVQLVESGGGLVQDGGSLRLACVASRSTRDINSMGWYRQAPGEQREFVASIGWQGATVYADSVEGRFTISRDDAKNTLYLQMNSLKPEDTAVYYCGADWRTYGYFYWGQGTQVTVS

SEQ ID NO: 14 - protein, 114 aa, Lama glama
QLVESGGGLVQDGDSLRLACAASATTVDINSMGWYRQAPGKQRELVASINTRGCTVYTDSVEGRFIIYRDDTKNTLYLQMYSLKSEDTAVYYCGADWRTNGYFYWGQGTQVIVS

SEQ ID NO: 15 - protein, 116 aa, Lama glama
QVQLVESGGGLIQDGGSLRLACVASRSTRGINSMGWYRQAPGEQREFVASIGWQGATVYADSVEGRFTISRDDAKNTLYLEMNSLNPEDTAVYYCGADWRTSGYFYWGQGTQVIVS

SEQ ID NO: 16 - protein, 124 aa, Lama glama
QVQLVESGGGLVQAGGSLRLSCAASGRITSYYGVGWFRQAPGKGREFVAVVTWNAGITFYADSVKGRFTISRDNAKNTVYLQMNGLKPEDTAVYYCAAGVFRARGYTLSGEYEYWGQGTQVTVS

SEQ ID NO: 17 - protein, 117 aa, Lama glama
QVQLVESGGGLVQAGGSLRLSCAASGIIFNTHTMAWYRQAPGKQRELVGRITFTGRTIYILDAVKGRFSISRNTADNTLTLQMNSLKPEDTAVYYCQTRNIRNNNENWGQGTQVTVS

SEQ ID NO: 18 - protein, 117 aa, Lama glama
QVQLVESGGGLVQAGGSLRLSCAASGRTLSRYAISWFRQAPGKEREFVGRITWSGSTNIADSVKGRFTISRDNAKNTVYLQMNSLKPEDTAVYYCAAAPYGTMSYDYWGQGTQVSVS

SEQ ID NO: 19 - protein, 113 aa, Lama glama
QVQFVESGGGLVQAGGSLRLSCVASGRAFSTYTIGWYRRPPGKQRELVATIGGNGNTYYVGSAKGRFTISRDNAKNTVYLQMNSLKPEDTDVYYCNRLGALDTWGQGTQVTVS

SEQ ID NO: 20 - protein, 114 aa, Lama glama
QVQLVESGGGLVQAGGSLRLSCVASGRIFSPNDMGWYRQVPGKQRELVAGMSSRGFTQYAESMEGRVTISRNNAENTAYLQMNGLQPDDTAVYYCYLWTEGHNFWGQGTQVTVS

SEQ ID NO: 21 - protein, 114 aa, Lama glama
QVQLVESGGGLVQAGGSLRLSCVASGKIFSPNDMGWYRQVPGKQRELVAAMSSRGFTNYAESLTDRFTVSRDNAKNTVYLQMNGLKPDDTAVYYCYLWTAGDNFWGQGTQVTVS